(19) United States
(12) Patent Application Publication
(10) Pub. No.: US 2012/0316455 A1
(43) Pub. Date: Dec. 13, 2012

(54) WEARABLE DEVICE AND PLATFORM FOR SENSORY INPUT

(75) Inventors: Hosain Sadequr Rahman, San Francisco, CA (US); Richard Lee Drysdale, Santa Cruz, CA (US); Michael Edward Smith Luna, San Jose, CA (US); Scott Fullam, Palo Alto, CA (US); Travis Austin Bogard, San Francisco, CA (US); Jeremiah Robison, San Francisco, CA (US); Max Everett Utter, II, San Francisco, CA (US); Thomas Alan Donaldson, London (GB); Raymond A. Martino, Los Gatos, CA (US)

(73) Assignee: AliphCom, San Francisco, CA (US)

(21) Appl. No.: 13/181,498

(22) Filed: Jul. 12, 2011

Related U.S. Application Data
(63) Continuation-in-part of application No. 13/180,000, filed on Jul. 11, 2011.
Application No. 13/180,000 is a continuation-in-part of application No. 13/180,320, filed on Jul. 11, 2011.
Application No. 13/180,320 is a continuation-in-part of application No. 13/158,372, filed on Jun. 10, 2011.
Application No. 13/158,372 is a continuation-in-part of application No. 13/158,416, filed on Jun. 11, 2011.
(60) Provisional application No. 61/495,995, filed on Jun. 11, 2011.
(60) Provisional application No. 61/495,994, filed on Jun. 11, 2011.
(60) Provisional application No. 61/495,997, filed on Jun. 11, 2011.
(60) Provisional application No. 61/495,996, filed on Jun. 11, 2011.

(51) Int. Cl. A61B 5/00; G01C 21/20; A61B 5/053; A61B 5/11; A61B 5/01

(52) U.S. Cl. 600/547; 600/300; 600/595; 600/549

(57) ABSTRACT

Techniques for a wearable device and platform for sensory input are described, including a sensor coupled to a framework having a housing having one or more moldings, the sensor being configured to sense at least one sensory input, a processor configured to transform the at least one sensory input to data during an activity in which the wearable device is worn, and a communications facility coupled to the wearable device and configured to transfer the data between the wearable device and another device during the activity, the data being configured to be presented on a user interface.

### CROSS-REFERENCE TO RELATED APPLICATIONS

This application is a continuation-in-part U.S. non-provisional patent application of U.S. patent application Ser. No. 13/180,000, filed Jul. 11, 2011, entitled “Data-Capable Band for Medical Diagnosis, Monitoring, and Treatment,” U.S. patent application Ser. No. 13/180,320, filed Jul. 11, 2011, entitled “Power Management in a Data-Capable Strapband,” U.S. patent application Ser. No. 13/158,372; filed Jun. 10, 2011, and entitled “Component Protective Overmolding,” U.S. patent application Ser. No. 13/158,416, filed Jun. 11, 2011, and entitled “Component Protective Overmolding,” and claims benefit to U.S. Provisional Patent Application No. 61/495,995, filed Jun. 11, 2011, and entitled “Data-Capable Strapband,” U.S. Provisional Patent Application No. 61,495,994, filed Jun. 11, 2011, and entitled “Data-Capable Strapband,” U.S. Provisional Patent Application No. 61/495,997, filed Jun. 11, 2011, and entitled “Data-Capable Strapband,” and U.S. Provisional Patent Application No. 61/495,996, filed Jun. 11, 2011, and entitled “Data-Capable Strapband,” all of which are herein incorporated by reference for all purposes.

### FIELD

The present invention relates generally to electrical and electronic hardware, computer software, wired and wireless network communications, and computing devices. More specifically, techniques for a wearable device and platform for sensory input are described.

### BACKGROUND

With the advent of greater computing capabilities in smaller personal and/or portable form factors and an increasing number of applications (i.e., computer and Internet software or programs) for different uses, consumers (i.e., users) have access to large amounts of personal data. Information and data are often readily available, but poorly captured using conventional data capture devices. Conventional devices typically lack capabilities that can capture, analyze, communicate, or use data in a contextually-meaningful, comprehensive, and efficient manner. Further, conventional solutions are often limited to specific individual purposes or uses, demanding that users invest in multiple devices in order to perform different activities (e.g., a sports watch for tracking time and distance, a GPS receiver for monitoring a hike or run, a cyclometer for gathering cycling data, and others). Although a wide range of data and information is available, conventional devices and applications fail to provide effective solutions that comprehensively capture data for a given user across numerous disparate activities.

Some conventional solutions combine a small number of discrete functions. Functionality for data capture, processing, storage, or communication in conventional devices such as a watch or timer with a heart rate monitor or global positioning system (“GPS”) receiver are available conventionally, but are expensive to manufacture and purchase. Other conventional solutions for combining personal data capture facilities often present numerous design and manufacturing problems such as size restrictions, specialized materials requirements, lowered tolerances for defects such as pits or holes in coverings for water-resistant or waterproof devices, unreliability, higher failure rates, increased manufacturing time, and expense. Subsequently, conventional devices such as fitness watches, heart rate monitors, GPS-enabled fitness monitors, health monitors (e.g., diabetic blood sugar testing units), digital voice recorders, pedometers, altimeters, and other conventional personal data capture devices are generally manufactured for conditions that occur in a single or small groupings of activities.

Generally, if the number of activities performed by conventional personal data capture devices increases, there is a corresponding rise in design and manufacturing requirements that results in significant consumer expense, which eventually becomes prohibitive to both investment and commercialization. Further, conventional manufacturing techniques are often limited and ineffective at meeting increased requirements to protect sensitive hardware, circuitry, and other components that are susceptible to damage, but which are required to perform various personal data capture activities. As a conventional example, sensitive electronic components such as printed circuit board assemblies (“PCBA”), sensors, and computer memory (hereafter “memory”) can be significantly damaged or destroyed during manufacturing processes where overmoldings or layering of protective material occurs using techniques such as injection molding, cold molding, and others. Damaged or destroyed items subsequently raises the cost of goods sold and can deter not only investment and commercialization, but also innovation in data capture and analysis technologies, which are highly compelling fields of opportunity.

Thus, what is needed is a solution for data capture devices without the limitations of conventional techniques.

### BRIEF DESCRIPTION OF THE DRAWINGS

Various embodiments or examples (“examples”) are disclosed in the following detailed description and the accompanying drawings:

FIG. 1 illustrates an exemplary data-capable strapband system;

FIG. 2A illustrates an exemplary wearable device and platform for sensory input;

FIG. 2B illustrates an alternative exemplary wearable device and platform for sensory input;

FIG. 3 illustrates sensors for use with an exemplary data-capable strapband;

FIG. 4 illustrates an application architecture for an exemplary data-capable strapband;

FIG. 5A illustrates representative data types for use with an exemplary data-capable strapband;

FIG. 5B illustrates representative data types for use with an exemplary data-capable strapband in fitness-related activities;

FIG. 5C illustrates representative data types for use with an exemplary data-capable strapband in sleep management activities;

FIG. 5D illustrates representative data types for use with an exemplary data-capable strapband in medical-related activities;

FIG. 5E illustrates representative data types for use with an exemplary data-capable strapband in social media/networking-related activities;

FIG. 6 illustrates a transition between modes of operation of a strapband in accordance with various embodiments;

FIG. 7A illustrates a perspective view of an exemplary data-capable strapband;

FIG. 7B illustrates a side view of an exemplary data-capable strapband;

FIG. 7C illustrates another side view of an exemplary data-capable strapband;

FIG. 7D illustrates a top view of an exemplary data-capable strapband;

FIG. 7E illustrates a bottom view of an exemplary data-capable strapband;

FIG. 7F illustrates a front view of an exemplary data-capable strapband;

FIG. 7G illustrates a rear view of an exemplary data-capable strapband;

FIG. 8A illustrates a perspective view of an exemplary data-capable strapband;

FIG. 8B illustrates a side view of an exemplary data-capable strapband;

FIG. 8C illustrates another side view of an exemplary data-capable strapband;

FIG. 8D illustrates a top view of an exemplary data-capable strapband;

FIG. 8E illustrates a bottom view of an exemplary data-capable strapband;

FIG. 8F illustrates a front view of an exemplary data-capable strapband;

FIG. 8G illustrates a rear view of an exemplary data-capable strapband;

FIG. 9A illustrates a perspective view of an exemplary data-capable strapband;

FIG. 9B illustrates a side view of an exemplary data-capable strapband;

FIG. 9C illustrates another side view of an exemplary data-capable strapband;

FIG. 9D illustrates a top view of an exemplary data-capable strapband;

FIG. 9E illustrates a bottom view of an exemplary data-capable strapband;

FIG. 9F illustrates a front view of an exemplary data-capable strapband;

FIG. 9G illustrates a rear view of an exemplary data-capable strapband;

FIG. 10 illustrates an exemplary computer system suitable for use with a data-capable strapband;

FIG. 11 depicts a variety of inputs in a specific example of a strapband, such as a data-capable strapband, according to various embodiments;

FIGS. 12A to 12F depict a variety of motion signatures as input into a strapband, such as a data-capable strapband, according to various embodiments;

FIG. 13 depicts an inference engine of a strapband configured to detect an activity and/or a mode based on monitored motion, according to various embodiments;

FIG. 14 depicts a representative implementation of one or more strapbands and equivalent devices, as wearable devices, to form unique motion profiles, according to various embodiments;

FIG. 15 depicts an example of a motion capture manager configured to capture motion and portions thereof, according to various embodiments;

FIG. 16 depicts an example of a motion analyzer configured to evaluate motion-centric events, according to various embodiments;

FIG. 17 illustrates action and event processing during a mode of operation in accordance with various embodiments;

FIG. 18A illustrates an exemplary wearable device for sensory user interface;

FIG. 18B illustrates an alternative exemplary wearable device for sensory user interface;

FIG. 18C illustrates an exemplary switch rod to be used with an exemplary wearable device;

FIG. 18D illustrates an exemplary switch for use with an exemplary wearable device; and

FIG. 18E illustrates an exemplary sensory user interface.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

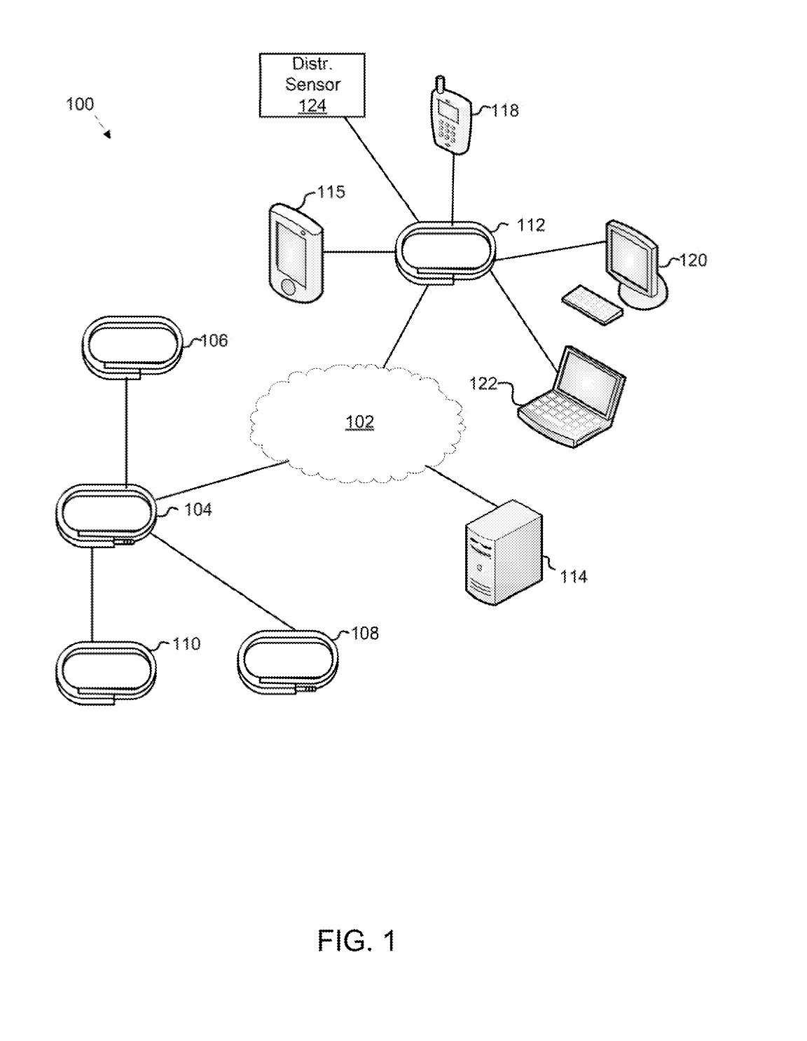

FIG. 1 illustrates an exemplary data-capable strapband system. Here, system 100 includes network 102, strapbands (hereafter “bands”) 104-112, server 114, mobile computing device 115, mobile communications device 118, computer 120, laptop 122, and distributed sensor 124. Although used interchangeably, “strapband” and “band” may be used to refer to the same or substantially similar data-capable device that may be worn as a strap or band around an arm, leg, ankle, or other bodily appendage or feature. In other examples, bands 104-112 may be attached directly or indirectly to other items, organic or inorganic, animate, or static. In still other examples, bands 104-112 may be used differently.

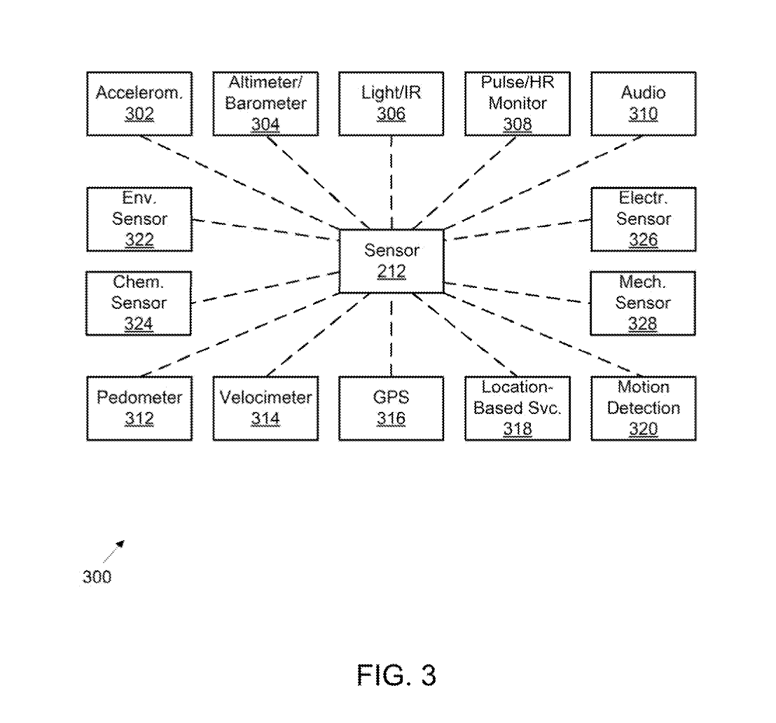

As described above, bands 104-112 may be implemented as wearable personal data or data capture devices (e.g., data-capable devices) that are worn by a user around a wrist, ankle, arm, ear, or other appendage, or attached to the body or affixed to clothing. One or more facilities, sensing elements, or sensors, both active and passive, may be implemented as part of bands 104-112 in order to capture various types of data from different sources. Temperature, environmental, temporal, motion, electronic, electrical, chemical, or other types of sensors (including those described below in connection with FIG. 3) may be used in order to gather varying amounts of data, which may be configurable by a user, locally (e.g., using user interface facilities such as buttons, switches, motion-activated/detected command structures (e.g., accelerometer-gathered data from user-initiated motion of bands 104-112), and others) or remotely (e.g., entering rules or parameters in a website or graphical user interface (“GUI”) that may be used to modify control systems or signals in firmware, circuitry, hardware, and software implemented (i.e., installed) on bands 104-112). In some examples, a user interface may be any type of human-computing interface (e.g., graphical, visual, audible, haptic, or any other type of interface that communicates information to a user (i.e., wearer of bands 104-112) using, for example, noise, light, vibration, or other sources of energy and data generation (e.g., pulsing vibrations to represent various types of signals or meanings, blinking lights, and the like, without limitation)) implemented locally (i.e., on or coupled to one or more of bands 104-112) or remotely (i.e., on a device other than bands 104-112). In other examples, a wearable device such as bands 104-112 may also be implemented as a user interface configured to receive and provide input to or from a user (i.e., wearer). Bands 104-112 may also be implemented as data-capable devices that are configured for data communication using various types of communications infrastructure and media, as described in greater detail below. Bands 104-112 may also be wearable, personal, non-intrusive, lightweight devices that are configured to gather large amounts of personally relevant data that can be used to improve user health, fitness levels, medical conditions, athletic performance, sleeping physiology, and physiological conditions, or used as a sensory-based user interface (“UI”) to signal social-related notifications specifying the state of the user through vibration, heat, lights or other sensory based notifications. For example, a social-related notification signal indicating a user is on-line can be transmitted to a recipient, who in turn, receives the notification as, for instance, a vibration.

Using data gathered by bands 104-112, applications may be used to perform various analyses and evaluations that can generate information as to a person's physical (e.g., healthy, sick, weakened, or other states, or activity level), emotional, or mental state (e.g., an elevated body temperature or heart rate may indicate stress, a lowered heart rate and skin temperature, or reduced movement (excessive sleeping), may indicate physiological depression caused by exertion or other factors, chemical data gathered from evaluating outgassing from the skin's surface may be analyzed to determine whether a person's diet is balanced or if various nutrients are lacking, salinity detectors may be evaluated to determine if high, lower, or proper blood sugar levels are present for diabetes management, and others). Generally, bands 104-112 may be configured to gather from sensors locally and remotely.

As an example, band 104 may capture (i.e., record, store, communicate (i.e., send or receive), process, or the like) data from various sources (i.e., sensors that are organic (i.e., installed, integrated, or otherwise implemented with band 104) or distributed (e.g., microphones on mobile computing device 115, mobile communications device 118, computer 120, laptop 122, distributed sensor 124, global positioning system (“GPS”) satellites (in low, mid, or high earth orbit), or others, without limitation)) and exchange data with one or more of bands 106-112, server 114, mobile computing device 115, mobile communications device 118, computer 120, laptop 122, and distributed sensor 124. As shown here, a local sensor may be one that is incorporated, integrated, or otherwise implemented with bands 104-112. A remote or distributed sensor (e.g., mobile computing device 115, mobile communications device 118, computer 120, laptop 122, or, generally, distributed sensor 124) may be sensors that can be accessed, controlled, or otherwise used by bands 104-112. For example, band 112 may be configured to control devices that are also controlled by a given user (e.g., mobile computing device 115, mobile communications device 118, computer 120, laptop 122, and distributed sensor 124). For example, a microphone in mobile communications device 118 may be used to detect, for example, ambient audio data that is used to help identify a person's location, or an ear clip (e.g., a headset as described below) affixed to an ear may be used to record pulse or blood oxygen saturation levels. Additionally, a sensor implemented with a screen on mobile computing device 115 may be used to read a user's temperature or obtain a biometric signature while a user is interacting with data. A further example may include using data that is observed on computer 120 or laptop 122 that provides information as to a user's online behavior and the type of content that she is viewing, which may be used by bands 104-112. Regardless of the type or location of sensor used, data may be transferred to bands 104-112 by using, for example, an analog audio jack, digital adapter (e.g., USB, mini-USB), or other, without limitation, plug, or other type of connector that may be used to physically couple bands 104-112 to another device or system for transferring data and, in some examples, to provide power to recharge a battery (not shown). Alternatively, a wireless data communication interface or facility (e.g., a wireless radio that is configured to communicate data from bands 104-112 using one or more data communication protocols (e.g., IEEE 802.11a/b/g/n (WiFi), WiMax, ANT™, ZigBee®, Bluetooth®, Near Field Communications (“NFC”), and others)) may be used to receive or transfer data. Further, bands 104-112 may be configured to analyze, evaluate, modify, or otherwise use data gathered, either directly or indirectly.

In some examples, bands 104-112 may be configured to share data with each other or with an intermediary facility, such as a database, website, web service, or the like, which may be implemented by server 114. In some embodiments, server 114 can be operated by a third party providing, for example, social media-related services. Bands 104-112 and other related devices may exchange data with each other directly, or bands 104-112 may exchange data via a third party server, such as a third party like Facebook®, to provide social-media related services. Examples of third party servers include servers for social networking services, including, but not limited to, services such as Facebook®, Yahoo! IM™, GTaIk™, MSN Messenger™, Twitter® and other private or public social networks. The exchanged data may include personal 20 physiological data and data derived from sensory-based user interfaces (“UI”). Server 114, in some examples, may be implemented using one or more processor-based computing devices or networks, including computing clouds, storage area networks (“SAN”), or the like. As shown, bands 104-112 may be used as a personal data or area network (e.g., “PDN” or “PAN”) in which data relevant to a given user or band (e.g., one or more of bands 104-112) may he shared. As shown here, bands 104 and 112 may be configured to exchange data with each other over network 102 or indirectly using server 114. Users of bands 104 and 112 may direct a web browser hosted on a computer (e.g., computer 120, laptop 122, or the like) in order to access, view, modify, or perform other operations with data captured by bands 104 and 112. For example, two runners using bands 104 and 112 may be geographically remote (e.g., users are not geographically in close proximity locally such that bands being used by each user are in direct data communication), but wish to share data regarding their race times (pre, post, or in-race), personal records (i.e., “PR”), target split times, results, performance characteristics (e.g., target heart rate, target VO₂ max, and others), and other information. If both runners (i.e., bands 104 and 112) are engaged in a race on the same day, data can be gathered for comparative analysis and other uses. Further, data can be shared in substantially real-time (taking into account any latencies incurred by data transfer rates, network topologies, or other data network factors) as well as uploaded after a given activity or event has been performed. In other words, data can be captured by the user as it is worn and configured to transfer data using, for example, a wireless network connection (e.g., a wireless network interface card, wireless local area network (“LAN”) card, cell phone, or the like. Data may also be shared in a temporally asynchronous manner in which a wired data connection (e.g., an analog audio plug (and associated software or firmware) configured to transfer digitally encoded data to encoded audio data that may be transferred between bands 104-112 and a plug configured to receive, encode/decode, and process data exchanged) may be used to transfer data from one or more bands 104-112 to various destinations (e.g., another of bands 104-112, server 114, mobile computing device 115, mobile communications device 118, computer 120, laptop 122, and distributed sensor 124). Bands 104-112 may be implemented with various types of wired and/or wireless communication facilities and are not intended to be limited to any specific technology. For example, data may be transferred from bands 104-112 using an analog audio plug (e.g., TRRS, TRS, or others). In other examples, wireless communication facilities using various types of data communication protocols (e.g., WiFi, Bluetooth®, ZigBee®, ANT™, and others) may be implemented as part of bands 104-112, which may include circuitry, firmware, hardware, radios, antennas, processors, microprocessors, memories, or other electrical, electronic, mechanical, or physical elements configured to enable data communication capabilities of various types and characteristics.

As data-capable devices, bands 104-112 may be configured to collect data from a wide range of sources, including onboard (not shown) and distributed sensors (e.g., server 114, mobile computing device 115, mobile communications device 118, computer 120, laptop 122, and distributed sensor 124) or other bands. Some or all data captured may be personal, sensitive, or confidential and various techniques for providing secure storage and access may be implemented. For example, various types of security protocols and algorithms may be used to encode data stored or accessed by bands 104-112. Examples of security protocols and algorithms include authentication, encryption, encoding, private and public key infrastructure, passwords, checksums, hash codes and hash functions (e.g., SHA, SHA-1, MD-5, and the like), or others may be used to prevent undesired access to data captured by bands 104-112. In other examples, data security for bands 104-112 may be implemented differently.

Bands 104-112 may be used as personal wearable, data capture devices that, when worn, are configured to identify a specific, individual user. By evaluating captured data such as motion data from an accelerometer, biometric data such as heart rate, skin galvanic response, and other biometric data, and using analysis techniques, both long and short-term (e.g., software packages or modules of any type, without limitation), a user may have a unique pattern of behavior or motion and/or biometric responses that can be used as a signature for identification. For example, bands 104-112 may gather data regarding an individual person's gait or other unique biometric, physiological or behavioral characteristics. Using, for example, distributed sensor 124, a biometric signature (e.g., fingerprint, retinal or iris vascular pattern, or others) may be gathered and transmitted to bands 104-112 that, when combined with other data, determines that a given user has been properly identified and, as such, authenticated. When bands 104-112 are worn, a user may be identified and authenticated to enable a variety of other functions such as accessing or modifying data, enabling wired or wireless data transmission facilities (i.e., allowing the transfer of data from bands 104-112), modifying functionality or functions of bands 104-112, authenticating financial transactions using stored data and information (e.g., credit card, PIN, card security numbers, and the like), running applications that allow for various operations to be performed (e.g., controlling physical security and access by transmitting a security code to a reader that, when authenticated, unlocks a door by turning off current to an electromagnetic lock, and others), and others. Different functions and operations beyond those described may be performed using bands 104-112, which can act as secure, personal, wearable, data-capable devices. The number, type, function, configuration, specifications, structure, or other features of system 100 and the above-described elements may be varied and are not limited to the examples provided.

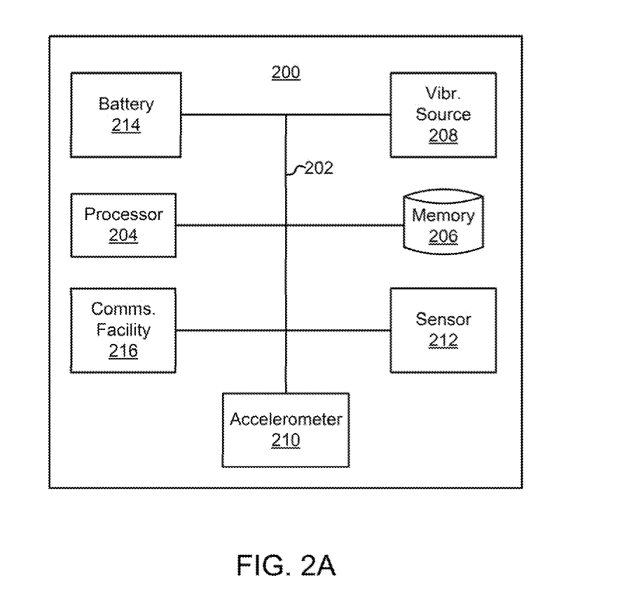

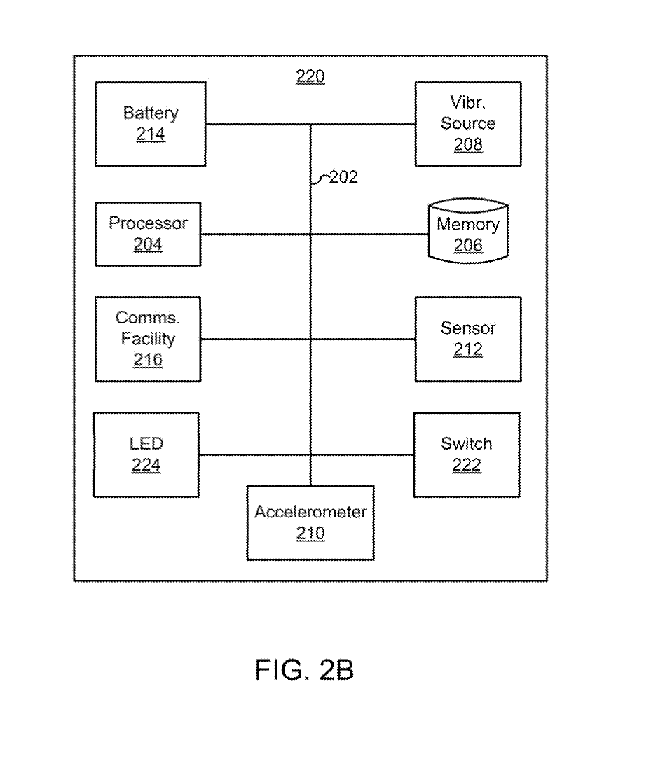

FIG. 2A illustrates an exemplary wearable device and platform for sensory input. Here, band (i.e., wearable device) 200 includes bus 202, processor 204, memory 206, vibration source 208, accelerometer 210, sensor 212, battery 214, and communications facility 216. In some examples, the quantity, type, function, structure, and configuration of band 200 and the elements (e.g., bus 202, processor 204, memory 206, vibration source 208, accelerometer 210, sensor 212, battery 214, and communications facility 216) shown may be varied and are not limited to the examples provided. As shown, processor 204 may be implemented as logic to provide control functions and signals to memory 206, vibration source 208, accelerometer 210, sensor 212, battery 214, and communications facility 216. Processor 204 may be implemented using any type of processor or microprocessor suitable for packaging within bands 104-112 (FIG. 1). Various types of microprocessors may be used to provide data processing capabilities for band 200 and are not limited to any specific type or capability. For example, a MSP430F5528-type microprocessor manufactured by Texas Instruments of Dallas, Tex. may be configured for data communication using audio tones and enabling the use of an audio plug-and-jack system (e.g., TRRS, TRS, or others) for transferring data captured by band 200. Further, different processors may be desired if other functionality (e.g., the type and number of sensors (e.g., sensor 212)) are varied. Data processed by processor 204 may be stored using, for example, memory 206.

In some examples, memory 206 may be implemented using various types of data storage technologies and standards, including, without limitation, read-only memory (“ROM”), random access memory (“RAM”), dynamic random access memory (“DRAM”), static random access memory (“SRAM”), static/dynamic random access memory (“SDRAM”), magnetic random access memory (“MRAM”), solid state, two and three-dimensional memories, Flash®, and others. Memory 206 may also be implemented using one or more partitions that are configured for multiple types of data storage technologies to allow for non-modifiable (i.e., by a user) software to be installed (e.g., firmware installed on ROM) while also providing for storage of captured data and applications using, for example, RAM. Once captured and/or stored in memory 206, data may be subjected to various operations performed by other elements of band 200.

Vibration source 208, in some examples, may be implemented as a motor or other mechanical structure that functions to provide vibratory energy that is communicated through band 200. As an example, an application stored on memory 206 may be configured to monitor a clock signal from processor 204 in order to provide timekeeping functions to band 200. If an alarm is set for a desired time, vibration source 208 may be used to vibrate when the desired time occurs. As another example, vibration source 208 may be coupled to a framework (not shown) or other structure that is used to translate or communicate vibratory energy throughout the physical structure of band 200. In other examples, vibration source 208 may be implemented differently.

Power may be stored in battery 214, which may be implemented as a battery, battery module, power management module, or the like. Power may also be gathered from local power sources such as solar panels, thermo-electric generators, and kinetic energy generators, among others that are alternatives power sources to external power for a battery. These additional sources can either power the system directly or can charge a battery, which, in turn, is used to power the system (e.g., of a strapband). In other words, battery 214 may include a rechargeable, expendable, replaceable, or other type of battery, but also circuitry, hardware, or software that may be used in connection with in lieu of processor 204 in order to provide power management, charge/recharging, sleep, or other functions. Further, battery 214 may be implemented using various types of battery technologies, including Lithium Ion (“LI”), Nickel Metal Hydride (“NiMH”), or others, without limitation. Power drawn as electrical current may be distributed from battery via bus 202, the latter of which may be implemented as deposited or formed circuitry or using other forms of circuits or cabling, including flexible circuitry. Electrical current distributed from battery 204 and managed by processor 204 may be used by one or more of memory 206, vibration source 208, accelerometer 210, sensor 212, or communications facility 216.

As shown, various sensors may be used as input sources for data captured by band 200. For example, accelerometer 210 may be used to detect a motion or other condition and convert it to data as measured across one, two, or three axes of motion. In addition to accelerometer 210, other sensors (i.e., sensor 212) may be implemented to provide temperature, environmental, physical, chemical, electrical, or other types of sensory inputs. As presented here, sensor 212 may include one or multiple sensors and is not intended to be limiting as to the quantity or type of sensor implemented. Sensory input captured by band 200 using accelerometer 210 and sensor 212 or data requested from another source (i.e., outside of band 200) may also be converted to data and exchanged, transferred, or otherwise communicated using communications facility 216. As used herein, “facility” refers to any, some, or all of the features and structures that are used to implement a given set of functions. For example, communications facility 216 may include a wireless radio, control circuit or logic, antenna, transceiver, receiver, transmitter, resistors, diodes, transistors, or other elements that are used to transmit and receive data from band 200. In some examples, communications facility 216 may be implemented to provide a “wired” data communication capability such as an analog or digital attachment, plug, jack, or the like to allow for data to be transferred. In other examples, communications facility 216 may be implemented to provide a wireless data communication capability to transmit digitally encoded data across one or more frequencies using various types of data communication protocols, without limitation. In still other examples, band 200 and the above-described elements may be varied in function, structure, configuration, or implementation and are not limited to those shown and described.

FIG. 2B illustrates an alternative exemplary wearable device and platform for sensory input. Here, band (i.e., wearable device) 220 includes bus 202, processor 204, memory 206, vibration source 208, accelerometer 210, sensor 212, battery 214, communications facility 216, switch 222, and light-emitting diode (hereafter “LED”) 224. Like-numbered and named elements may be implemented similarly in function and structure to those described in prior examples. Further, the quantity, type, function, structure, and configuration of band 200 and the elements (e.g., bus 202, processor 204, memory 206, vibration source 208, accelerometer 210, sensor 212, battery 214, and communications facility 216) shown may be varied and are not limited to the examples provided.

In some examples, band 200 may be implemented as an alternative structure to band 200 (FIG. 2A) described above. For example, sensor 212 may be configured to sense, detect, gather, or otherwise receive input (i.e., sensed physical, chemical, biological, physiological, or psychological quantities) that, once received, may be converted into data and transferred to processor 204 using bus 202. As an example, temperature, heart rate, respiration rate, galvanic skin response (i.e., skin conductance response), muscle stiffness/fatigue, and other types of conditions or parameters may be measured using sensor 212, which may be implemented using one or multiple sensors. Further, sensor 212 is generally coupled (directly or indirectly) to band 220. As used herein, “coupled” may refer to a sensor being locally implemented on band 220 or remotely on, for example, another device that is in data communication with it.

Sensor 212 may be configured, in some examples, to sense various types of environmental (e.g., ambient air temperature, barometric pressure, location (e.g., using GPS or other satellite constellations for calculating Cartesian or other coordinates on the earth's surface, micro-cell network triangulation, or others), physical, physiological, psychological, or activity-based conditions in order to determine a state of a user of wearable device 220 (i.e., band 220). In other examples, applications or firmware may be downloaded that, when installed, may be configured to change sensor 212 in terms of function. Sensory input to sensor 212 may be used for various purposes such as measuring caloric burn rate, providing active (e.g., generating an alert such as vibration, audible, or visual indicator) or inactive (e.g., providing information, content, promotions, advertisements, or the like on a website, mobile website, or other location that is accessible using an account that is associated with a user and band 220) feedback, measuring fatigue (e.g., by calculating skin conductance response (hereafter “SCR”) using sensor 212 or accelerometer 210) or other physical states, determining a mood of a user, and others, without limitation. As used herein, feedback may be provided using a mechanism (i.e., feedback mechanism) that is configured to provide an alert or other indicator to a user. Various types of feedback mechanisms may be used, including a vibratory source, motor, light source (e.g., pulsating, blinking, or steady illumination), light emitting diode (e.g., LED 224), audible, audio, visual, haptic, or others, without limitation. Feedback mechanisms may provide sensory output of the types indicated above via band 200 or, in other examples, using other devices that may be in data communication with it. For example, a driver may receive a vibratory alert from vibration source (e.g., motor) 208 when sensor 212 detects skin tautness (using, for example, accelerometer to detect muscle stiffness) that indicates she is falling asleep and, in connection with a GPS-sensed signal, wearable device 220 determines that a vehicle is approaching a divider, intersection, obstacle, or is accelerating/decelerating rapidly, and the like. Further, an audible indicator may be generated and sent to an ear-worn communication device such as a Bluetooth® (or other data communication protocol, near or far field) headset. Other types of devices that have a data connection with wearable device 220 may also be used to provide sensory output to a user, such as using a mobile communications or computing device having a graphical user interface to display data or information associated with sensory input received by sensor 212.

In some examples, sensory output may be an audible tone, visual indication, vibration, or other indicator that can be provided by another device that is in data communication with band 220. In other examples, sensory output may be a media file such as a song that is played when sensor 212 detects a given parameter. For example, if a user is running and sensor 212 detects a heart rate that is lower than the recorded heart rate as measured against 65 previous runs, processor 204 may be configured to generate a control signal to an audio device that begins playing an upbeat or high tempo song to the user in order to increase her heart rate and activity-based performance. As another example, sensor 212 and/or accelerometer 210 may sense various inputs that can be measured against a calculated “lifeline” (e.g., LIFELINE™) that is an abstract representation of a user's health or wellness. If sensory input to sensor 212 (or accelerometer 210 or any other sensor implemented with band 220) is received, it may be compared to the user's lifeline or abstract representation (hereafter “representation”) in order to determine whether feedback, if any, should be provided in order to modify the user's behavior. A user may input a range of tolerance (i.e., a range within which an alert is not generated) or processor 204 may determine a range of tolerance to be stored in memory 206 with regard to various sensory input. For example, if sensor 212 is configured to measure internal bodily temperature, a user may set a 0.1 degree Fahrenheit range of tolerance to allow her body temperature to fluctuate between 98.5 and 98.7 degrees Fahrenheit before an alert is generated (e.g., to avoid heat stress, heat exhaustion, heat stroke, or the like). Sensor 212 may also be implemented as multiple sensors that are disposed (i.e., positioned) on opposite sides of band 220 such that, when worn on a wrist or other bodily appendage, allows for the measurement of skin conductivity in order to determine skin conductance response. Skin conductivity may be used to measure various types of parameters and conditions such as cognitive effort, arousal, lying, stress, physical fatigue due to poor sleep quality, emotional responses to various stimuli, and others.

Activity-based feedback may be given along with state-based feedback. In some examples, band 220 may be configured to provide feedback to a user in order to help him achieve a desired level of fitness, athletic performance, health, or wellness. In addition to feedback, band 220 may also be configured to provide indicators of use to a wearer during, before, or after a given activity or state.

As used herein, various types of indicators (e.g., audible, visual, mechanical, or the like) may also be used in order to provide a sensory user interface. In other words, band 220 may be configured with switch 222 that can be implemented using various types of structures as indicators of device state, function, operation, mode, or other conditions or characteristics. Examples of indicators include “wheel” or rotating structures such as dials or buttons that, when turned to a given position, indicate a particular function, mode, or state of band 220. Other structures may include single or multiple-position switches that, when turned to a given position, are also configured for the user to visually recognize a function, mode, or state of band 220. For example, a 4-position switch or button may indicate “on,” “off,” standby,” “active,” “inactive,” or other mode. A 2-position switch or button may also indicate other modes of operation such as “on” and “off.” As yet another example, a single switch or button may be provided such that, when the switch or button is depressed, band 220 changes mode or function without, alternatively, providing a visual indication. In other examples, different types of buttons, switches, or other user interfaces may be provided and are not limited to the examples shown.

FIG. 3 illustrates sensors for use with an exemplary data-capable strapband. Sensor 212 may be implemented using various types of sensors, some of which are shown. Like-numbered and named elements may describe the same or substantially similar element as those shown in other descriptions. Here, sensor 212 (FIG. 2) may be implemented as accelerometer 302, altimeter/barometer 304, light/infrared (“IR”) sensor 306, pulse/heart rate (“HR”) monitor 308, audio sensor (e.g., microphone, transducer, or others) 310, pedometer 312, velocimeter 314, GPS receiver 316, location-based service sensor (e.g., sensor for determining location within a cellular or micro-cellular network, which may or may not use GPS or other satellite constellations for fixing a position) 318, motion detection sensor 320, environmental sensor 322, chemical sensor 324, electrical sensor 326, or mechanical sensor 328.

As shown, accelerometer 302 may be used to capture data associated with motion detection along 1, 2, or 3-axes of measurement, without limitation to any specific type of specification of sensor. Accelerometer 302 may also be implemented to measure various types of user motion and may be configured based on the type of sensor, firmware, software, hardware, or circuitry used. As another example, altimeter/barometer 304 may be used to measure environment pressure, atmospheric or otherwise, and is not limited to any specification or type of pressure-reading device. In some examples, altimeter/barometer 304 may be an altimeter, a barometer, or a combination thereof. For example, altimeter/barometer 304 may be implemented as an altimeter for measuring above ground level (“AGL”) pressure in band 200, which has been configured for use by naval or military aviators. As another example, altimeter/barometer 304 may be implemented as a barometer for reading atmospheric pressure for marine-based applications. In other examples, altimeter/barometer 304 may be implemented differently.

Other types of sensors that may be used to measure light or photonic conditions include light/IR sensor 306, motion detection sensor 320, and environmental sensor 322, the latter of which may include any type of sensor for capturing data associated with environmental conditions beyond light. Further, motion detection sensor 320 may be configured to detect motion using a variety of techniques and technologies, including, but not limited to comparative or differential light analysis (e.g., comparing foreground and background lighting), sound monitoring, or others. Audio sensor 310 may be implemented using any type of device configured to record or capture sound.

In some examples, pedometer 312 may be implemented using devices to measure various types of data associated with pedestrian-oriented activities such as running or walking. Footstrikes, stride length, stride length or interval, time, and other data may be measured. Velocimeter 314 may be implemented, in some examples, to measure velocity (e.g., speed and directional vectors) without limitation to any particular activity. Further, additional sensors that may be used as sensor 212 include those configured to identify or obtain location-based data. For example, GPS receiver 316 may be used to obtain coordinates of the geographic location of band 200 using, for example, various types of signals transmitted by civilian and/or military satellite constellations in low, medium, or high earth orbit (e.g., “LEO,” “MEO,” or “GEO”). In other examples, differential GPS algorithms may also be implemented with GPS receiver 316, which may be used to generate more precise or accurate coordinates. Still further, location-based services sensor 318 may be implemented to obtain location-based data including, but not limited to location, nearby services or items of interest, and the like. As an example, location-based services sensor 318 may be configured to detect an electronic signal, encoded or otherwise, that provides information regarding a physical locale as band 200 passes. The electronic signal may include, in some examples, encoded data regarding the location and information associated therewith. Electrical sensor 326 and mechanical sensor 328 may be configured to include other types (e.g., haptic, kinetic, piezoelectric, piezomechanical, pressure, touch, thermal, and others) of sensors for data input to band 200, without limitation. Other types of sensors apart from those shown may also be used, including magnetic flux sensors such as solid-state compasses and the like. The sensors can also include gyroscopic sensors. While the present illustration provides numerous examples of types of sensors that may be used with band 200 (FIG. 2), others not shown or described may be implemented with or as a substitute for any sensor shown or described.

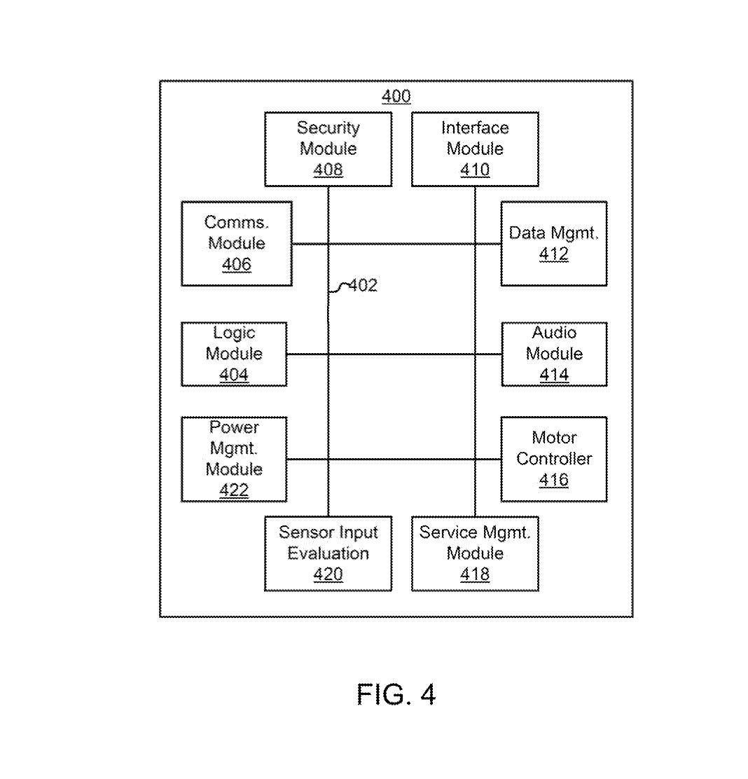

FIG. 4 illustrates an application architecture for an exemplary data-capable strapband. Here, application architecture 400 includes bus 402, logic module 404, communications module 406, security module 408, interface module 410, data management 412, audio module 414, motor controller 416, service management module 418, sensor input evaluation module 420, and power management module 422. In some examples, application architecture 400 and the above-listed elements (e.g., bus 402, logic module 404, communications module 406, security module 408, interface module 410, data management 412, audio module 414, motor controller 416, service management module 418, sensor input evaluation module 420, and power management module 422) may be implemented as software using various computer programming and formatting languages such as Java, C++, C, and others. As shown here, logic module 404 may be firmware or application software that is installed in memory 206 (FIG. 2) and executed by processor 204 (FIG. 2). Included with logic module 404 may be program instructions or code (e.g., source, object, binary executables, or others) that, when initiated, called, or instantiated, perform various functions.

For example, logic module 404 may be configured to send control signals to communications module 406 in order to transfer, transmit, or receive data stored in memory 206, the latter of which may be managed by a database management system (“DBMS”) or utility in data management module 412. As another example, security module 408 may be controlled by logic module 404 to provide encoding, decoding, encryption, authentication, or other functions to band 200 (FIG. 2). Alternatively, security module 408 may also be implemented as an application that, using data captured from various sensors and stored in memory 206 (and accessed by data management module 412) may be used to provide identification functions that enable band 200 to passively identify a user or wearer of band 200. Still further, various types of security software and applications may be used and are not limited to those shown and described.

Interface module 410, in some examples, may be used to manage user interface controls such as switches, buttons, or other types of controls that enable a user to manage various functions of band 200. For example, a 4-position switch may be turned to a given position that is interpreted by interface module 410 to determine the proper signal or feedback to send to logic module 404 in order to generate a particular result. In other examples, a button (not shown) may be depressed that allows a user to trigger or initiate certain actions by sending another signal to logic module 404. Still further, interface module 410 may be used to interpret data from, for example, accelerometer 210 (FIG. 2) to identify specific movement or motion that initiates or triggers a given response. In other examples, interface module 410 may be used to manage different types of displays (e.g., light-emitting diodes (LEDs), interferometric modulator display (IMOD), electrophoretic ink (E Ink), organic light-emitting diode (OLED), etc.). In other examples, interface module 410 may be implemented differently in function, structure, or configuration and is not limited to those shown and described.

As shown, audio module 414 may be configured to manage encoded or unencoded data gathered from various types of audio sensors. In some examples, audio module 414 may include one or more codecs that are used to encode or decode various types of audio waveforms. For example, analog audio input may be encoded by audio module 414 and, once encoded, sent as a signal or collection of data packets, messages, segments, frames, or the like to logic module 404 for transmission via communications module 406. In other examples, audio module 414 may be implemented differently in function, structure, configuration, or implementation and is not limited to those shown and described. Other elements that may be used by band 200 include motor controller 416, which may be firmware or an application to control a motor or other vibratory energy source (e.g., vibration source 208 (FIG. 2)). Power used for band 200 may be drawn from battery 214 (FIG. 2) and managed by power management module 422, which may be firmware or an application used to manage, with or without user input, how power is consumer, conserved, or otherwise used by band 200 and the above-described elements, including one or more sensors (e.g., sensor 212 (FIG. 2), sensors 302-328 (FIG. 3)). With regard to data captured, sensor input evaluation module 420 may be a software engine or module that is used to evaluate and analyze data received from one or more inputs (e.g., sensors 302-328) to band 200. When received, data may be analyzed by sensor input evaluation module 420, which may include custom or “off-the-shelf” analytics packages that are configured to provide application-specific analysis of data to determine trends, patterns, and other useful information. In other examples, sensor input module 420 may also include firmware or software that enables the generation of various types and formats of reports for presenting data and any analysis performed thereupon.

Another element of application architecture 400 that may be included is service management module 418. In some examples, service management module 418 may be firmware, software, or an application that is configured to manage various aspects and operations associated with executing software-related instructions for band 200. For example, libraries or classes that are used by software or applications on band 200 may be served from an online or networked source. Service management module 418 may be implemented to manage how and when these services are invoked in order to ensure that desired applications are executed properly within application architecture 400. As discrete sets, collections, or groupings of functions, services used by band 200 for various purposes ranging from communications to operating systems to call or document libraries may be managed by service management module 418. Alternatively, service management module 418 may be implemented differently and is not limited to the examples provided herein. Further, application architecture 400 is an example of a software/system/application-level architecture that may be used to implement various software-related aspects of band 200 and may be varied in the quantity, type, configuration, function, structure, or type of programming or formatting languages used, without limitation to any given example.

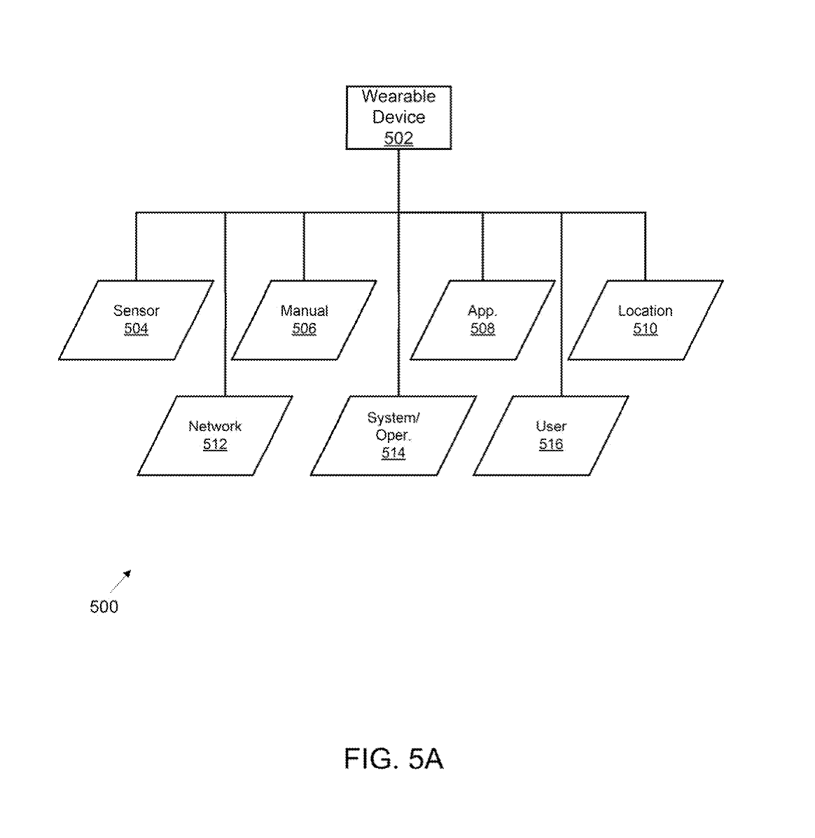

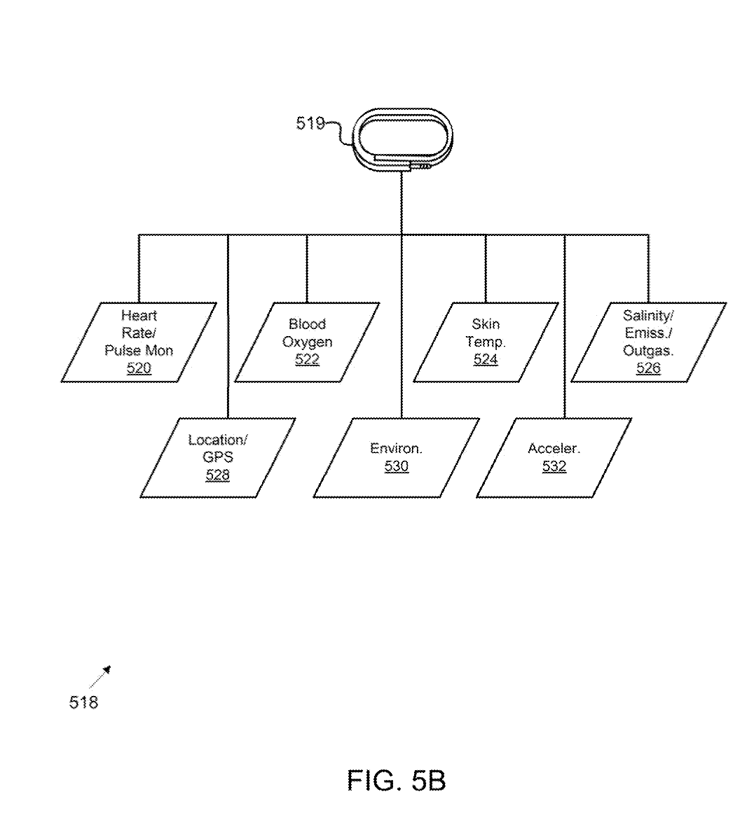

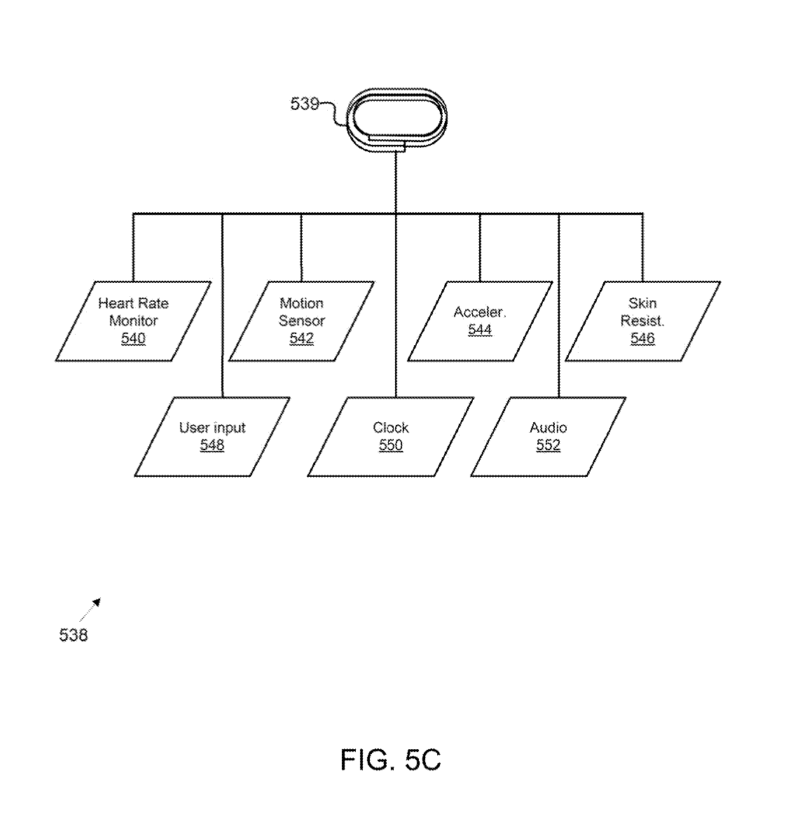

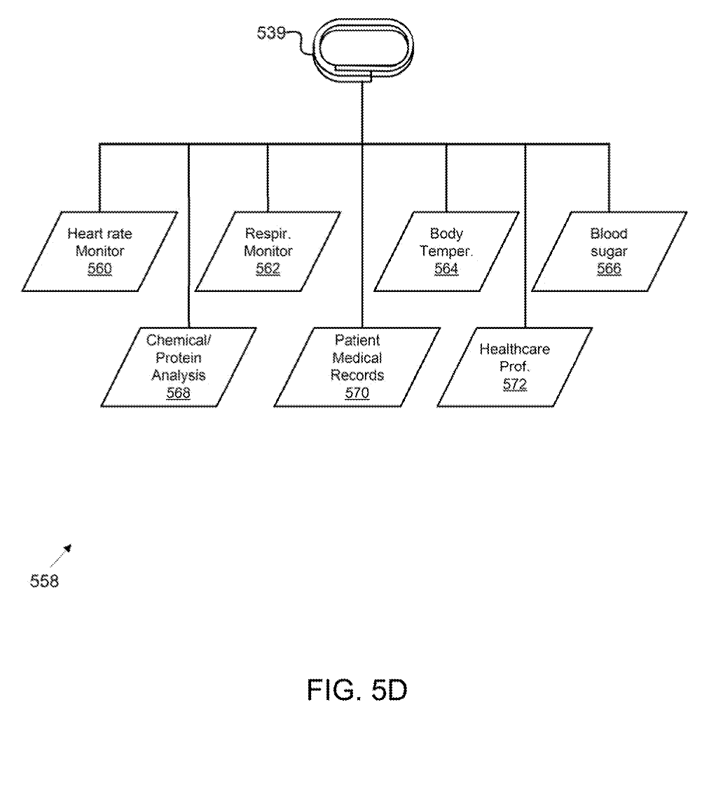

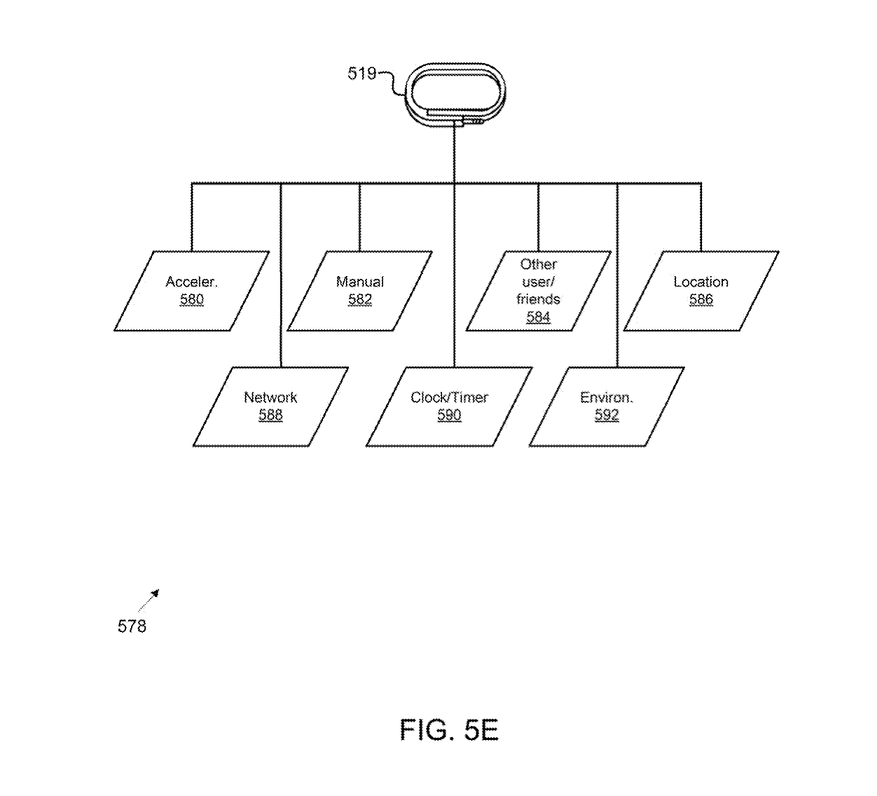

FIG. 5A illustrates representative data types for use with an exemplary data-capable strapband. Here, wearable device 502 may capture various types of data, including, but not limited to sensor data 504, manually-entered data 506, application data 508, location data 510, network data 512, system/operating data 514, and user data 516. Various types of data may be captured from sensors, such as those described above in connection with FIG. 3. Manually-entered data, in some examples, may be data or inputs received directly and locally by band 200 (FIG. 2). In other examples, manually-entered data may also be provided through a third-party website that stores the data in a database and may be synchronized from server 114 (FIG. 1) with one or more of bands 104-112. Other types of data that may be captured including application data 508 and system/operating data 514, which may be associated with firmware, software, or hardware installed or implemented on band 200. Further, location data 510 may be used by wearable device 502, as described above. User data 516, in some examples, may be data that include profile data, preferences, rules, or other information that has been previously entered by a given user of wearable device 502. Further, network data 512 may be data is captured by wearable device with regard to routing tables, data paths, network or access availability (e.g., wireless network access availability), and the like. Other types of data may be captured by wearable device 502 and are not limited to the examples shown and described. Additional context-specific examples of types of data captured by bands 104-112 (FIG. 1) are provided below.

FIG. 5B illustrates representative data types for use with an exemplary data-capable strapband in fitness-related activities. Here, band 519 may be configured to capture types (i.e., categories) of data such as heart rate/pulse monitoring data 520, blood oxygen saturation data 522, skin temperature data 524, salinity/emission/outgassing data 526, location/GPS data 528, environmental data 530, and accelerometer data 532. As an example, a runner may use or wear band 519 to obtain data associated with his physiological condition (i.e., heart rate/pulse monitoring data 520, skin temperature, salinity/emission/outgassing data 526, among others), athletic efficiency (i.e., blood oxygen saturation data 522), and performance (i.e., location/GPS data 528 (e.g., distance or laps run), environmental data 530 (e.g., ambient temperature, humidity, pressure, and the like), accelerometer 532 (e.g., biomechanical information, including gait, stride, stride length, among others)). Other or different types of data may be captured by band 519, but the above-described examples are illustrative of some types of data that may be captured by band 519. Further, data captured may be uploaded to a website or online/networked destination for storage and other uses. For example, fitness-related data may be used by applications that are downloaded from a “fitness marketplace” where athletes may find, purchase, or download applications for various uses. Some applications may be activity-specific and thus may be used to modify or alter the data capture capabilities of band 519 accordingly. For example, a fitness marketplace may be a website accessible by various types of mobile and non-mobile clients to locate applications for different exercise or fitness categories such as running, swimming, tennis, golf, baseball, football, fencing, and many others. When downloaded, a fitness marketplace may also be used with user-specific accounts to manage the retrieved applications as well as usage with band 519, or to use the data to provide services such as online personal coaching or targeted advertisements. More, fewer, or different types of data may be captured for fitness-related activities.

FIG. 5C illustrates representative data types for use with an exemplary data-capable strapband in sleep management activities. Here, band 539 may be used for sleep management purposes to track various types of data, including heart rate monitoring data 540, motion sensor data 542, accelerometer data 544, skin resistivity data 546, user input data 548, clock data 550, and audio data 552. In some examples, heart rate monitor data 540 may be captured to evaluate rest, waking, or various states of sleep. Motion sensor data 542 and accelerometer data 544 may be used to determine whether a user of band 539 is experiencing a restful or fitful sleep. For example, some motion sensor data 542 may be captured by a light sensor that measures ambient or differential light patterns in order to determine whether a user is sleeping on her front, side, or back. Accelerometer data 544 may also be captured to determine whether a user is experiencing gentle or violent disruptions when sleeping, such as those often found in afflictions of sleep apnea or other sleep disorders. Further, skin resistivity data 546 may be captured to determine whether a user is ill (e.g., running a temperature, sweating, experiencing chills, clammy skin, and others). Still further, user input data may include data input by a user as to how and whether band 539 should trigger vibration source 208 (FIG. 2) to wake a user at a given time or whether to use a series of increasing or decreasing vibrations to trigger a waking state. Clock data (550) may be used to measure the duration of sleep or a finite period of time in which a user is at rest. Audio data may also be captured to determine whether a user is snoring and, if so, the frequencies and amplitude therein may suggest physical conditions that a user may be interested in knowing (e.g., snoring, breathing interruptions, talking in one's sleep, and the like). More, fewer, or different types of data may be captured for sleep management-related activities.

FIG. 5D illustrates representative data types for use with an exemplary data-capable strapband in medical-related activities. Here, band 539 may also be configured for medical purposes and related-types of data such as heart rate monitoring data 560, respiratory monitoring data 562, body temperature data 564, blood sugar data 566, chemical protein/analysis data 568, patient medical records data 570, and healthcare professional (e.g., doctor, physician, registered nurse, physician's assistant, dentist, orthopedist, surgeon, and others) data 572. In some examples, data may be captured by band 539 directly from wear by a user. For example, band 539 may be able to sample and analyze sweat through a salinity or moisture detector to identify whether any particular chemicals, proteins, hormones, or other organic or inorganic compounds are present, which can be analyzed by band 539 or communicated to server 114 to perform further analysis. If sent to server 114, further analyses may be performed by a hospital or other medical facility using data captured by band 539. In other examples, more, fewer, or different types of data may be captured for medical-related activities.

FIG. 5E illustrates representative data types for use with an exemplary data-capable strapband in social media/networking-related activities. Examples of social media/networking-related activities include related to Internet-based Social Networking 15 Services (“SNS”), such as Facebook®, Twitter®, etc. Here, band 519, shown with an audio data plug, may be configured to capture data for use with various types of social media and networking-related services, websites, and activities. Accelerometer data 580, manual data 582, other user/friends data 584, location data 586, network data 588, clock/timer data 590, and environmental data 592 are examples of data that may be gathered and shared by, for example, uploading data from band 519 using, for example, an audio plug such as those described herein. As another example, accelerometer data 580 may be captured and shared with other users to share motion, activity, or other movement-oriented data. Manual data 582 may be data that a given user also wishes to share with other users. Likewise, other user/friends data 584 may be from other bands (not shown) that can be shared or aggregated with data captured by band 519. Location data 586 for band 519 may also be shared with other users. In other examples, a user may also enter manual data 582 to prevent other users or friends from receiving updated location data from band 519. Additionally, network data 588 and clock/timer data may be captured and shared with other users to indicate, for example, activities or events that a given user (i.e., wearing band 519) was engaged at certain locations. Further, if a user of band 519 has friends who are not geographically located in close or near proximity (e.g., the user of band 519 is located in San Francisco and her friend is located in Rome), environmental data can be captured by band 519 (e.g., weather, temperature, humidity, sunny or overcast (as interpreted from data captured by a light sensor and combined with captured data for humidity and temperature), among others). In other examples, more, fewer, or different types of data may be captured for medical-related activities.

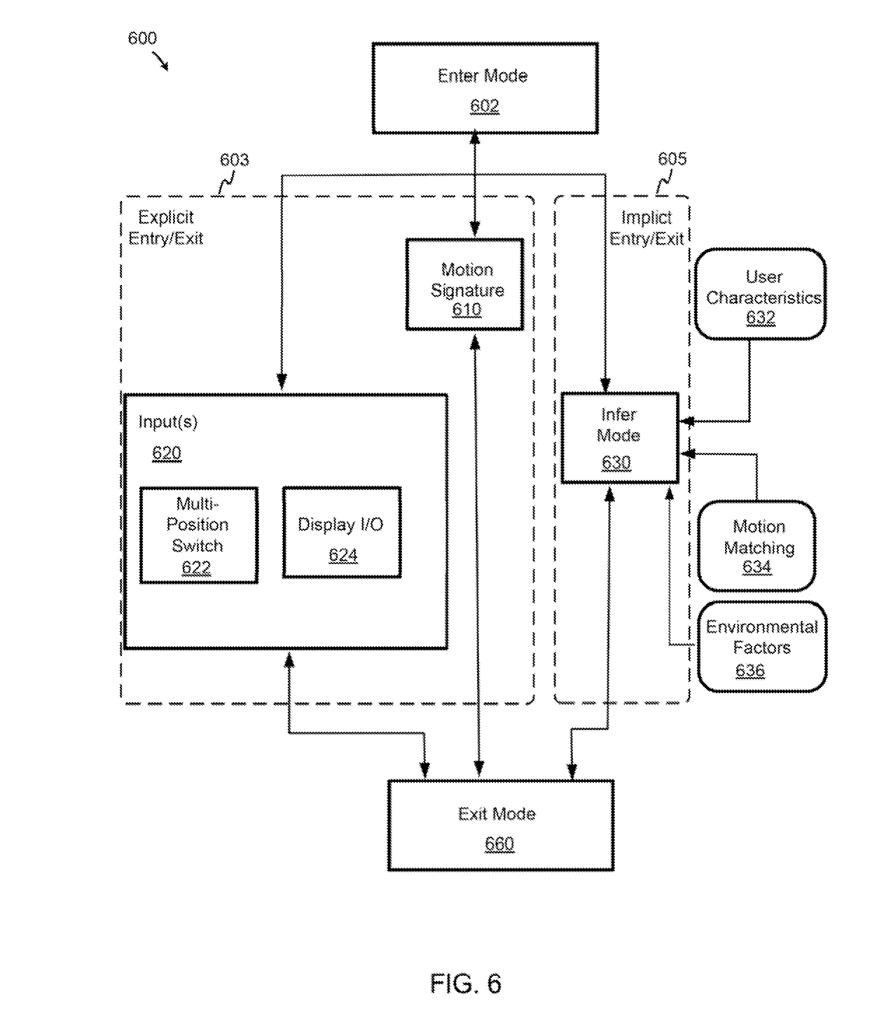

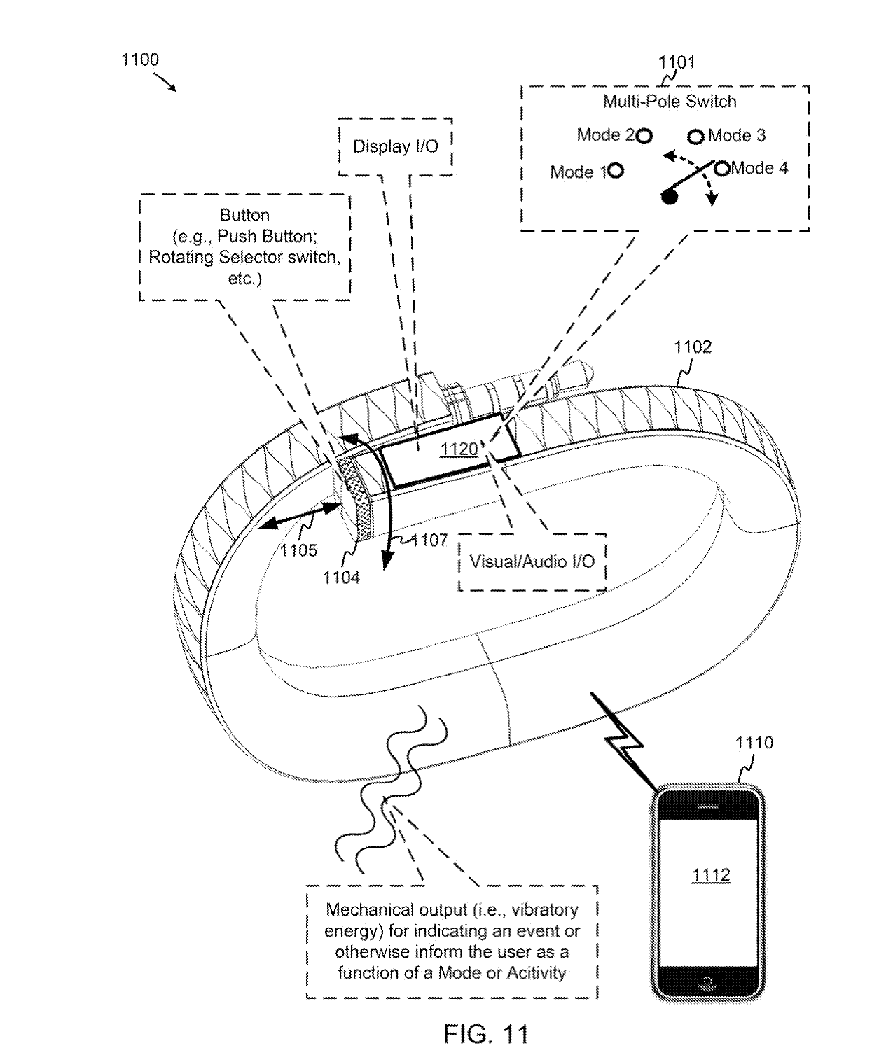

FIG. 6 illustrates a transition between modes of operation for a strapband in accordance with various embodiments. A strapband can transition between modes by either entering a mode at 602 or exiting a mode at 660. The flow to enter a mode begins at 602 and flows downward, whereas the flow to exit the mode begins at 660 and flows upward. A mode can be entered and exited explicitly 603 or entered and exited implicitly 605. In particular, a user can indicate explicitly whether to enter or exit a mode of operation by using inputs 620. Examples of inputs 620 include a switch with one or more positions that are each associated with a selectable mode, and a display I/O 624 that can be touch-sensitive for entering commands explicitly to enter or exit a mode. Note that entry of a second mode of operation can extinguish implicitly the first mode of operation. Further, a user can explicitly indicate whether to enter or exit a mode of operation by using motion signatures 610. That is, the motion of the strapband can facilitate transitions between modes of operation. A motion signature is a set of motions or patterns of motion that the strapband can detect using the logic of the strapband, whereby the logic can infer a mode from the motion signature. Examples of motion signatures are discussed below in FIG. 11. A set of motions can be predetermined, and then can be associated with a command to enter or exit a mode. Thus, motion can select a mode of operation. In some embodiments, modes of operation include a “normal” mode, an “active mode,” a “sleep mode” or “resting mode,”), among other types of modes. A normal mode includes usual or normative amount of activities, whereas, an “active mode” typically includes relatively large amounts of activity. Active mode can include activities, such as running and swimming, for example. A “sleep mode” or “resting mode” typically includes a relatively low amount of activity that is indicative of sleeping or resting can be indicative of the user sleeping.

A mode can be entered and exited implicitly 605. In particular, a strapband and its logic can determine whether to enter or exit a mode of operation by inferring either an activity or a mode at 630. An inferred mode of operation can be determined as a function of user characteristics 632, such as determined by user-relevant sensors (e.g., heart rate, body temperature, etc.). An inferred mode of operation can be determined using motion matching 634 (e.g., motion is analyzed and a type of activity is determined). Further, an inferred mode of operation can be determined by examining environmental factors 636 (e.g., ambient temperature, time, ambient light, etc.). To illustrate, consider that: (1.) user characteristics 632 specify that the user's heart rate is at a resting rate and the body temperature falls (indicative of resting or sleeping), (2.) motion matching 634 determines that the user has a relatively low level of activity, and (3.) environment factors 636 indicate that the time is 3:00 am and the ambient light is negligible. In view of the foregoing, an inference engine or other logic of the strapband likely can infer that the user is sleeping and then operate to transition the strapband into sleep mode. In this mode, power may be reduced. Note that while a mode may transition either explicitly or implicitly, it need not exit the same way.

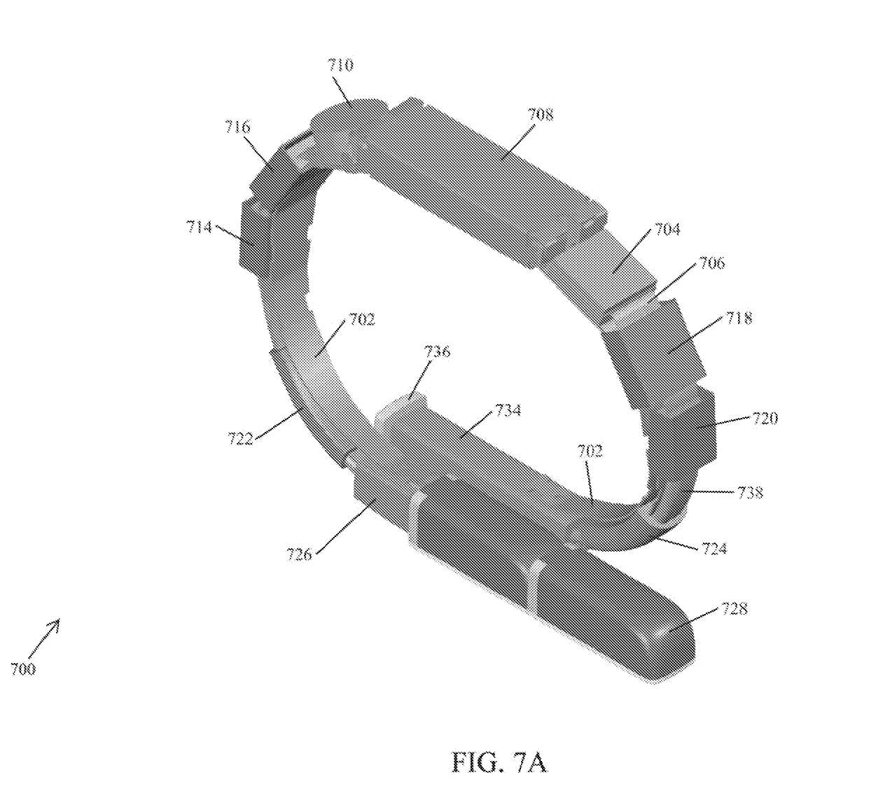

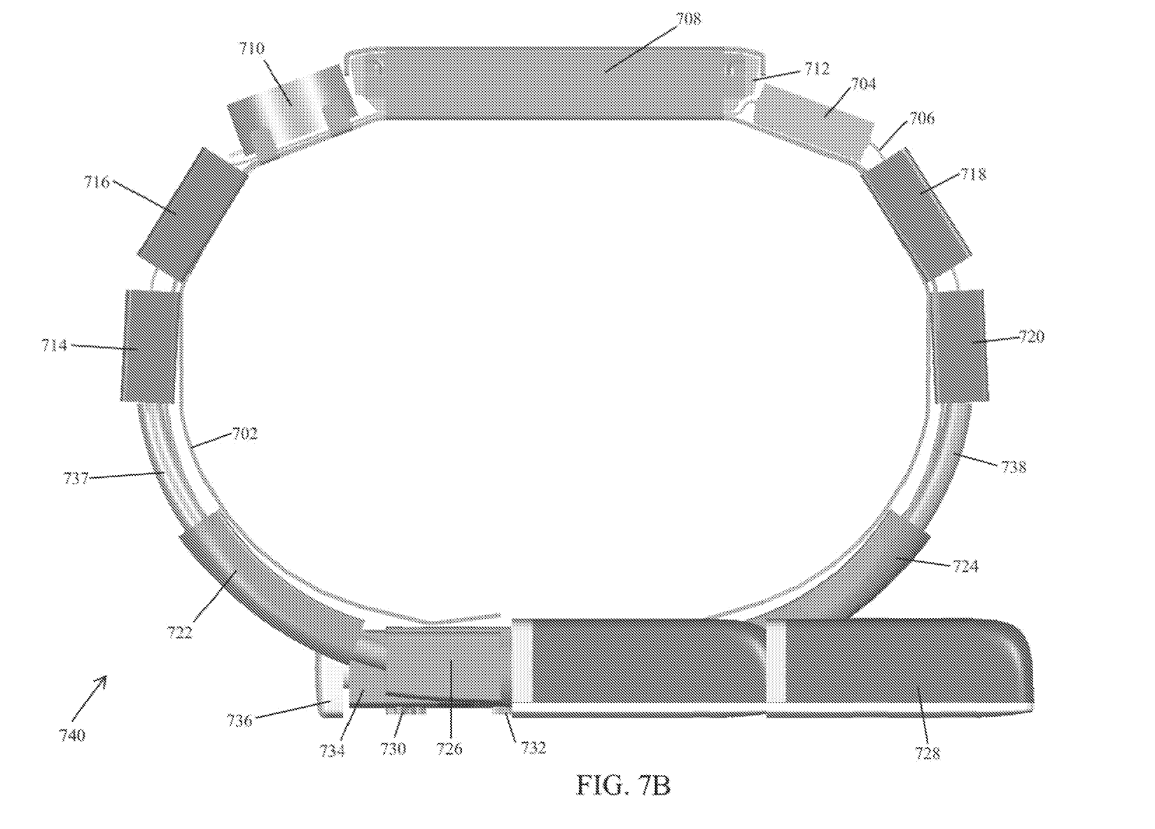

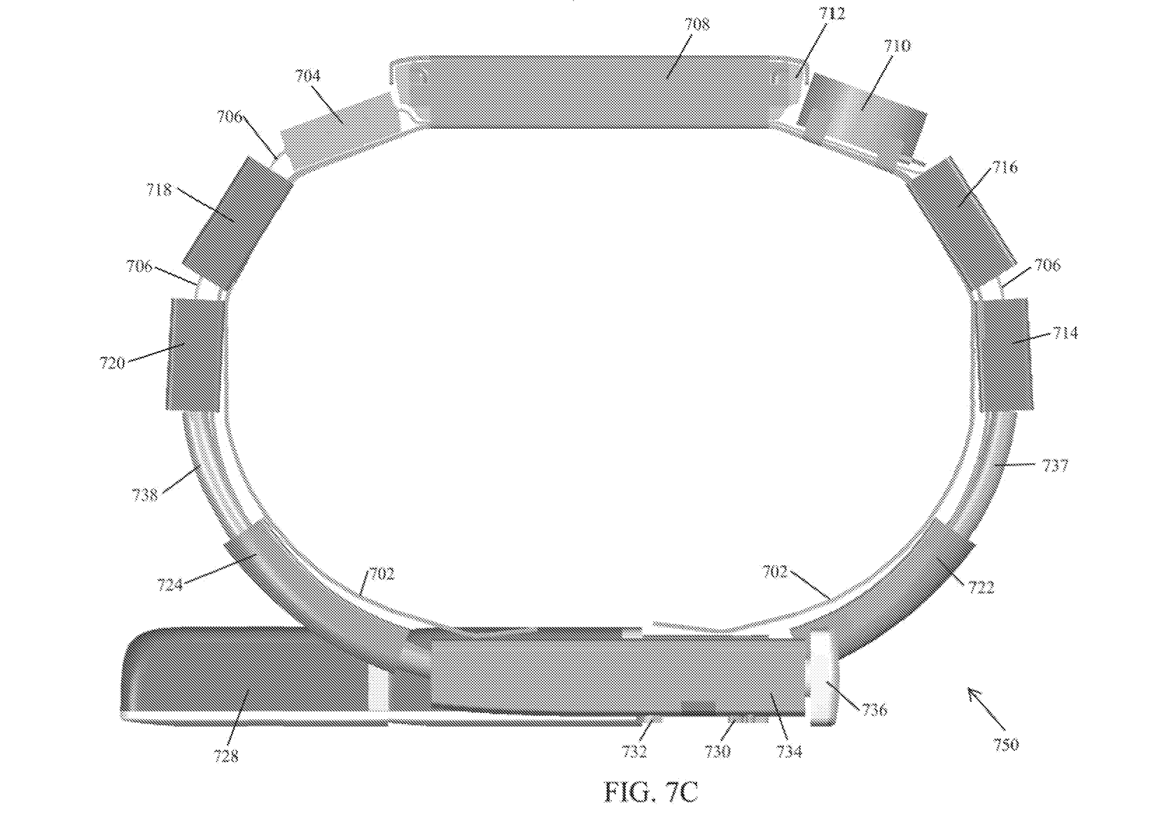

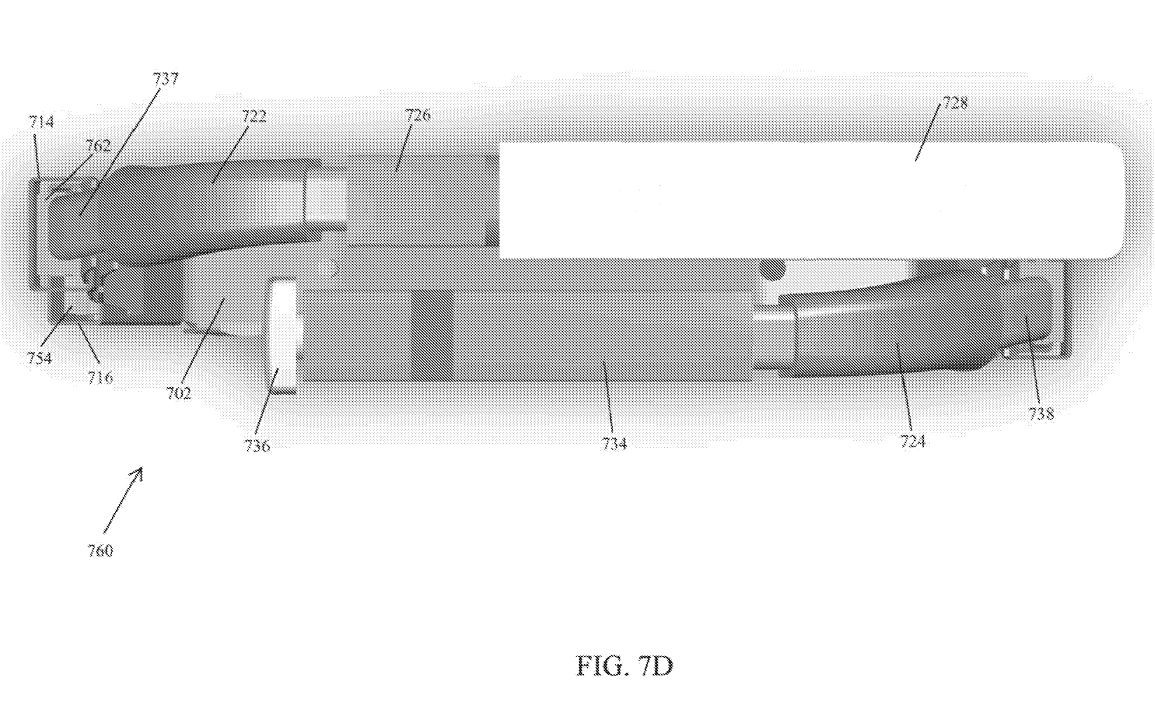

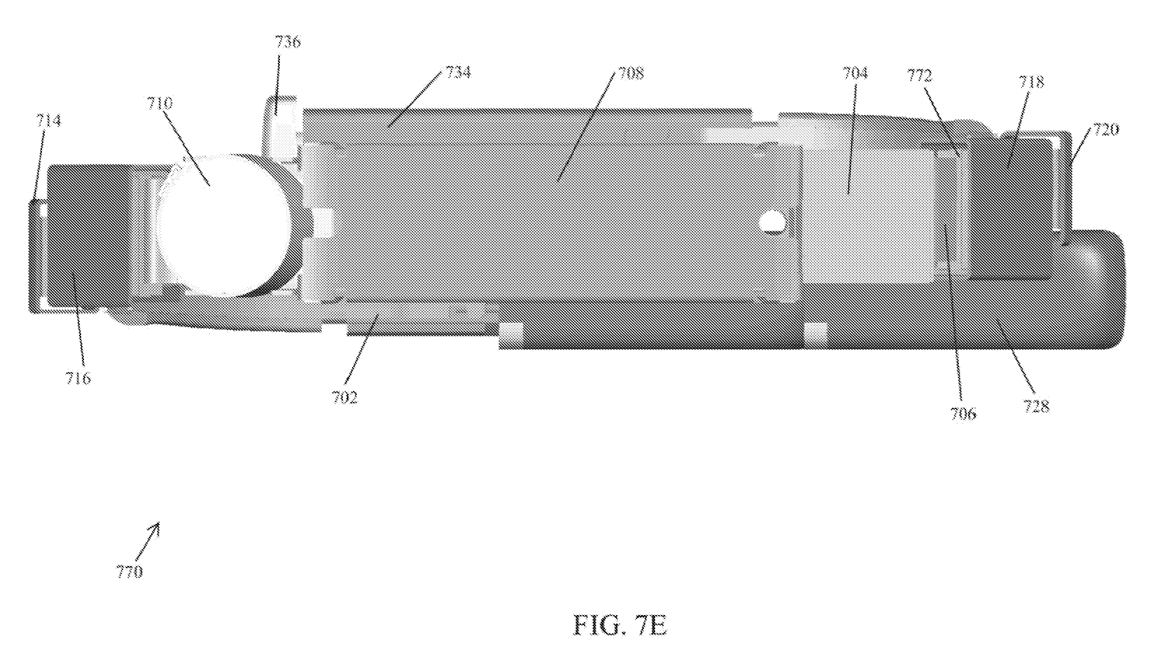

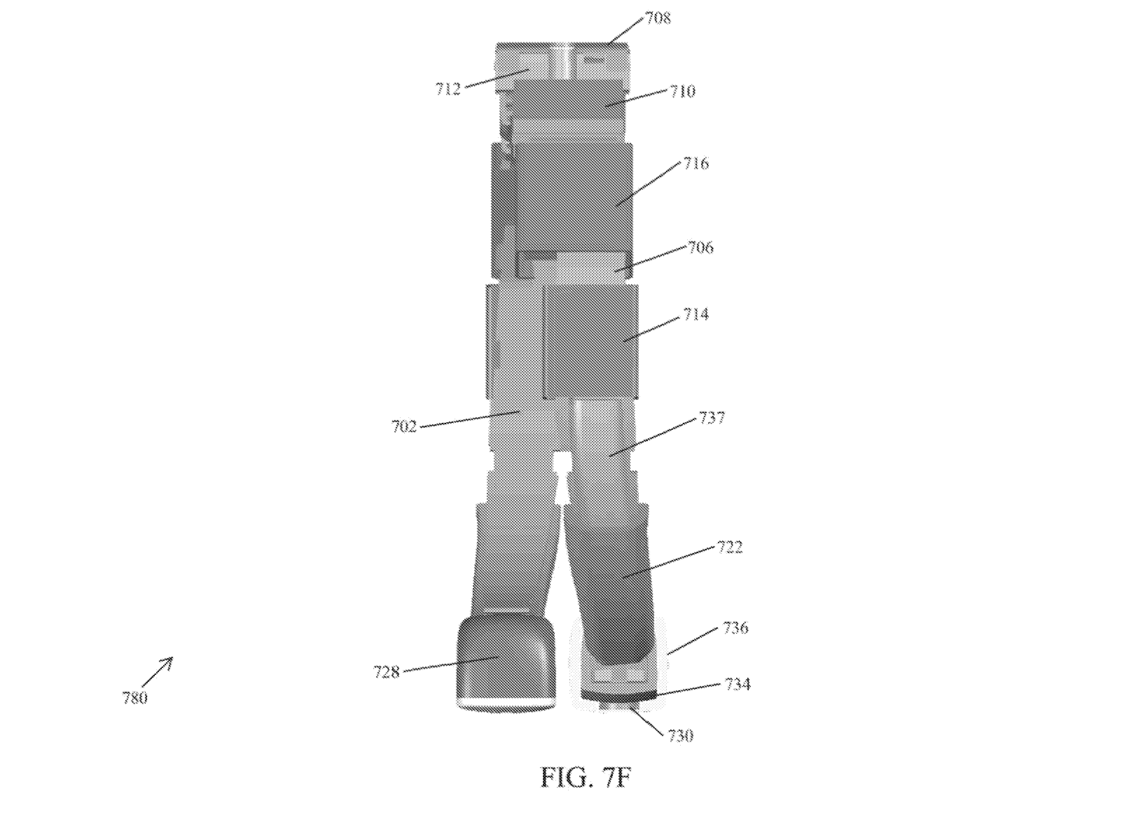

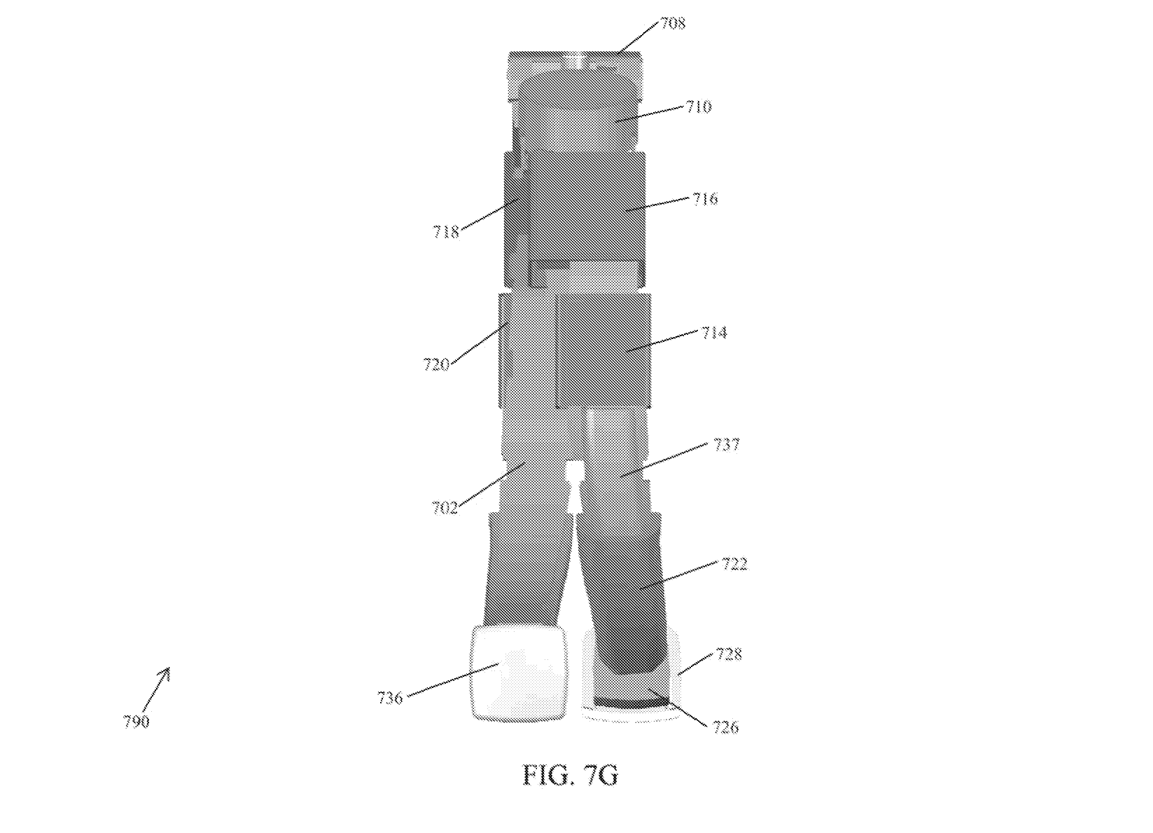

FIG. 7A illustrates a perspective view of an exemplary data-capable strapband configured to receive overmolding. Here, band 700 includes framework 702, covering 704, flexible circuit 706, covering 708, motor 710, coverings 714-724, plug 726, accessory 728, control housing 734, control 736, and flexible circuits 737-738. In some examples, band 700 is shown with various elements (i.e., covering 704, flexible circuit 706, covering 708, motor 710, coverings 714-724, plug 726, accessory 728, control housing 734, control 736, and flexible circuits 737-738) coupled to framework 702. Coverings 708, 714-724 and control housing 734 may be configured to protect various types of elements, which may be electrical, electronic, mechanical, structural, or of another type, without limitation. For example, covering 708 may be used to protect a battery and power management module from protective material formed around band 700 during an injection molding operation. As another example, housing 704 may be used to protect a printed circuit board assembly (“PCBA”) from similar damage. Further, control housing 734 may be used to protect various types of user interfaces (e.g., switches, buttons (e.g., control 736), lights, light-emitting diodes, or other control features and functionality) from damage. In other examples, the elements shown may be varied in quantity, type, manufacturer, specification, function, structure, or other aspects in order to provide data capture, communication, analysis, usage, and other capabilities to band 700, which may be worn by a user around a wrist, arm, leg, ankle, neck or other protrusion or aperture, without restriction. Band 700, in some examples, illustrates an initial unlayered device that may be protected using the techniques for protective overmolding as described above. Alternatively, the number, type, function, configuration, ornamental appearance, or other aspects shown may be varied without limitation.

FIG. 7B illustrates a side view of an exemplary data-capable strapband. Here, band 740 includes framework 702, covering 704, flexible circuit 706, covering 708, motor 710, battery 712, coverings 714-724, plug 726, accessory 728, button/switch/LED/LCD Display 730-732, control housing 734, control 736, and flexible circuits 737-738 and is shown as a side view of band 700. In other examples, the number, type, function, configuration, ornamental appearance, or other aspects shown may be varied without limitation.

FIG. 7C illustrates another side view of an exemplary data-capable strapband. Here, band 750 includes framework 702, covering 704, flexible circuit 706, covering 708, motor 710, battery 712, coverings 714-724, accessory 728, button/switch/LED/LCD Display 730-732, control housing 734, control 736, and flexible circuits 737-738 and is shown as an opposite side view of band 740. In some examples, button/switch/LED/LCD Display 730-732 may be implemented using different types of switches, including multiple position switches that may be manually turned to indicate a given function or command. Further, underlighting provided by light emitting diodes (“LED”) or other types of low power lights or lighting systems may be used to provide a visual status for band 750. In other examples, the number, type, function, configuration, ornamental appearance, or other aspects shown may be varied without limitation.

FIG. 7D illustrates a top view of an exemplary data-capable strapband. Here, hand 760 includes framework 702, coverings 714-716 and 722-724, plug 726, accessory 728, control housing 734, control 736, flexible circuits 737-738, and PCBA 762. In other examples, the number, type, function, configuration, ornamental appearance, or other aspects shown may be varied without limitation.

FIG. 7E illustrates a bottom view of an exemplary data-capable strapband. Here, band 770 includes framework 702, covering 704, flexible circuit 706, covering 708, motor 710, coverings 714-720, plug 726, accessory 728, control housing 734, control 736, and PCBA 772. In some examples, PCBA 772 may be implemented as any type of electrical or electronic circuit board element or component, without restriction. In other examples, the number, type, function, configuration, ornamental appearance, or other aspects shown may be varied without limitation.

FIG. 7F illustrates a front view of an exemplary data-capable strapband. Here, band 780 includes framework 702, flexible circuit 706, covering 708, motor 710, coverings 714-718 and 722, accessory 728, button/switch/LED/LCD Display 730, control housing 734, control 736, and flexible circuit 737. In some examples, button/switch/LED/LCD Display 730 may be implemented using various types of displays including liquid crystal (LCD), thin film, active matrix, and others, without limitation. In other examples, the number, type, function, configuration, ornamental appearance, or other aspects shown may be varied without limitation.

FIG. 7G illustrates a rear view of an exemplary data-capable strapband. Here, band 790 includes framework 702, covering 708, motor 710, coverings 714-722, analog audio plug 726, accessory 728, control 736, and flexible circuit 737. In some examples, control 736 may be a button configured for depression in order to activate or initiate other functionality of band 790. In other examples, the number, type, function, configuration, ornamental appearance, or other aspects shown may be varied without limitation.

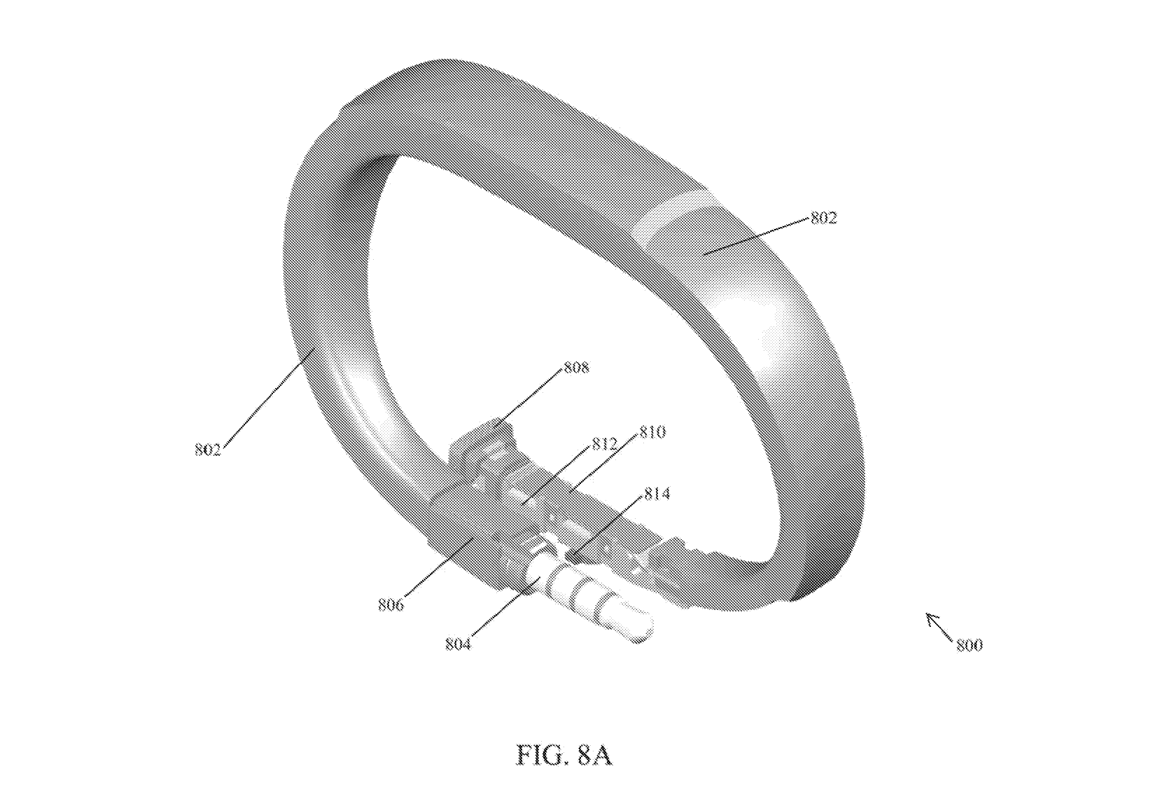

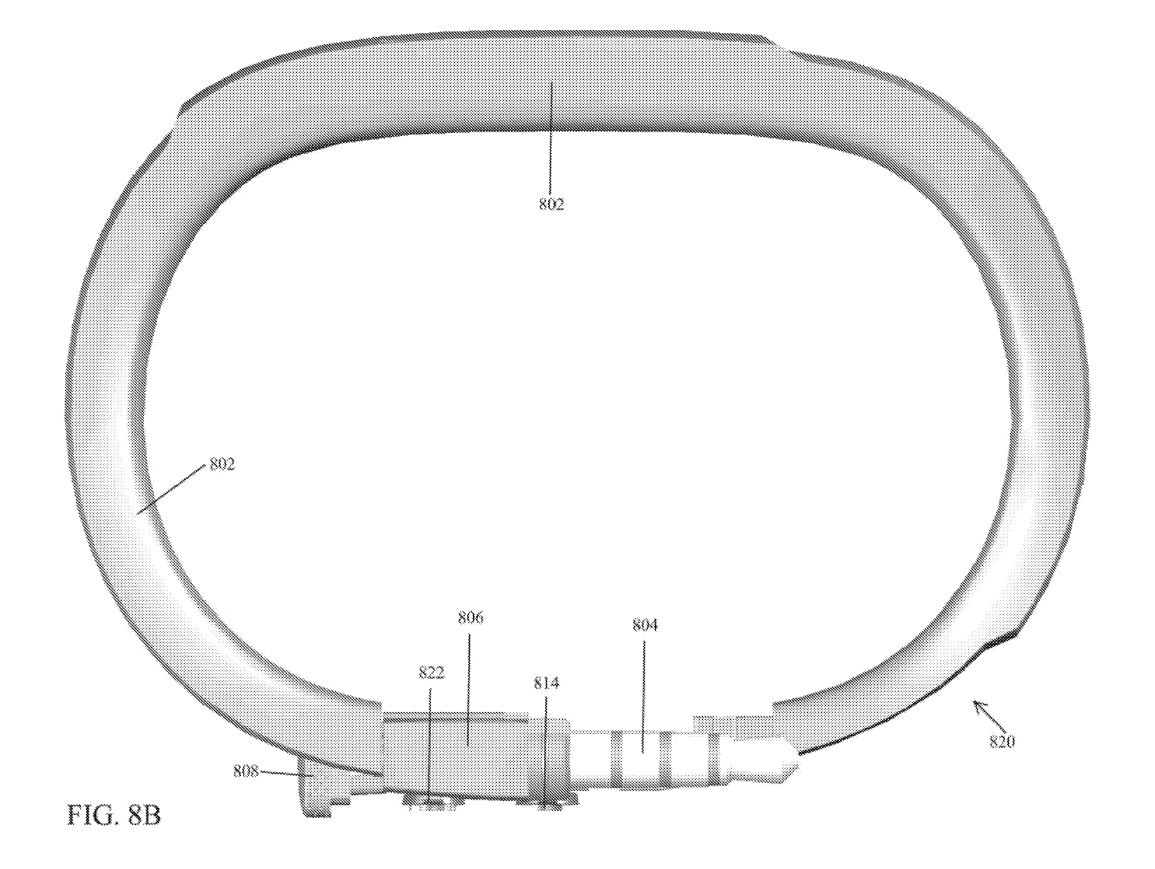

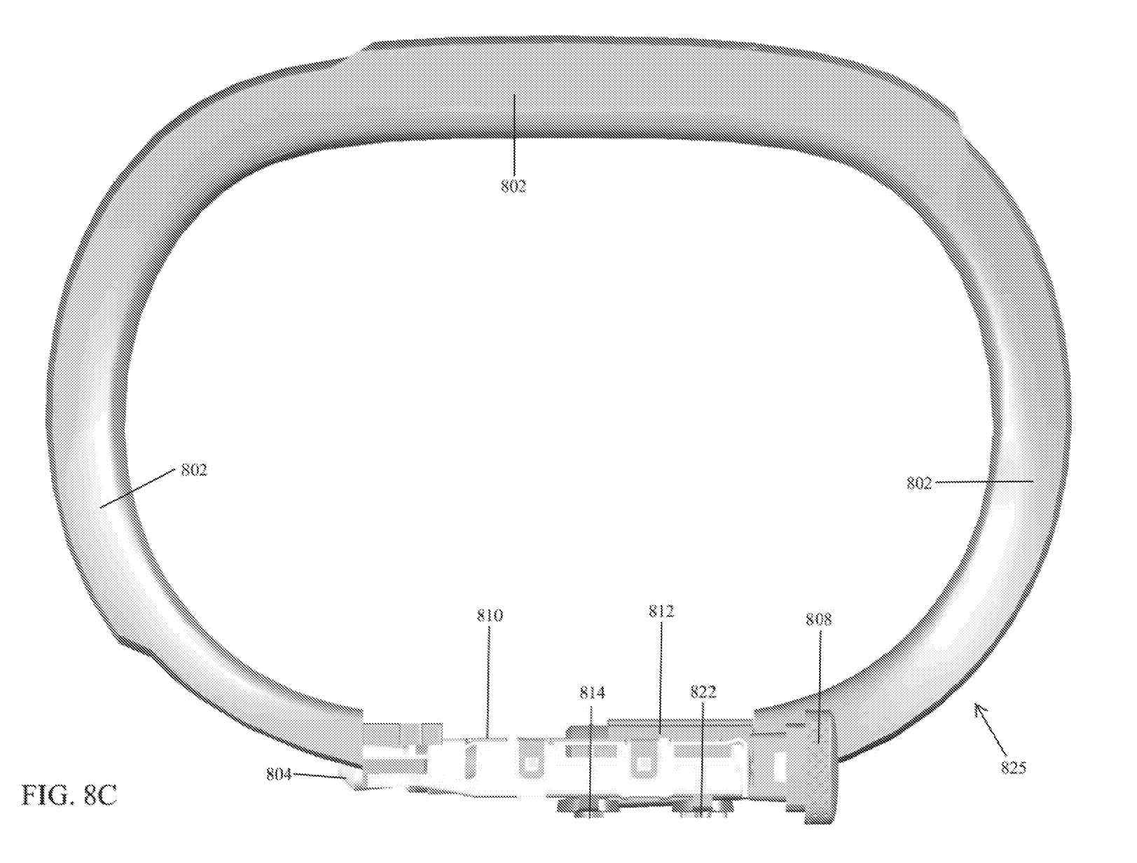

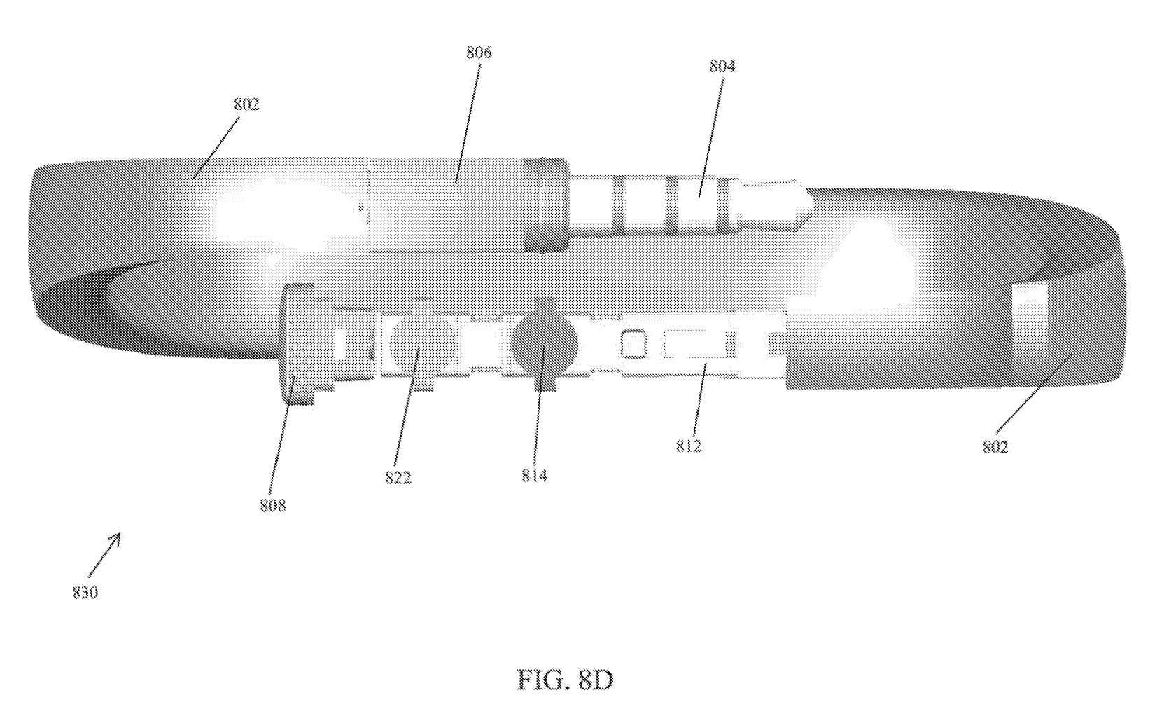

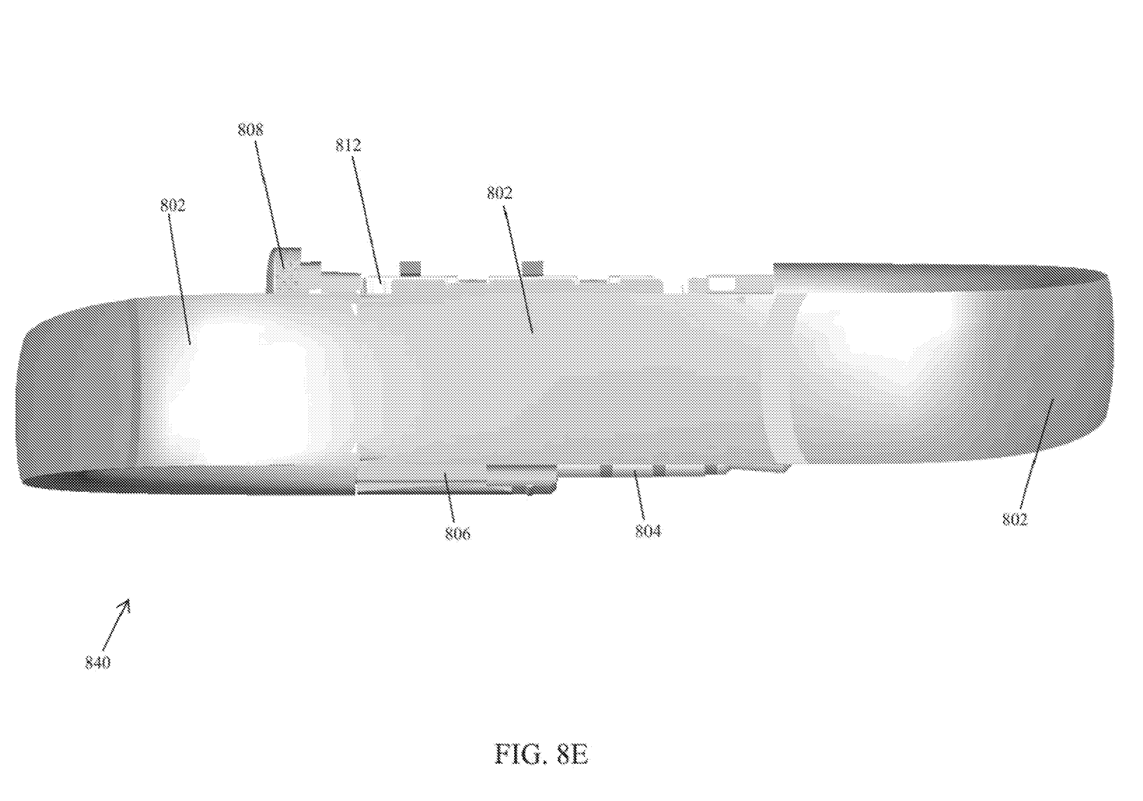

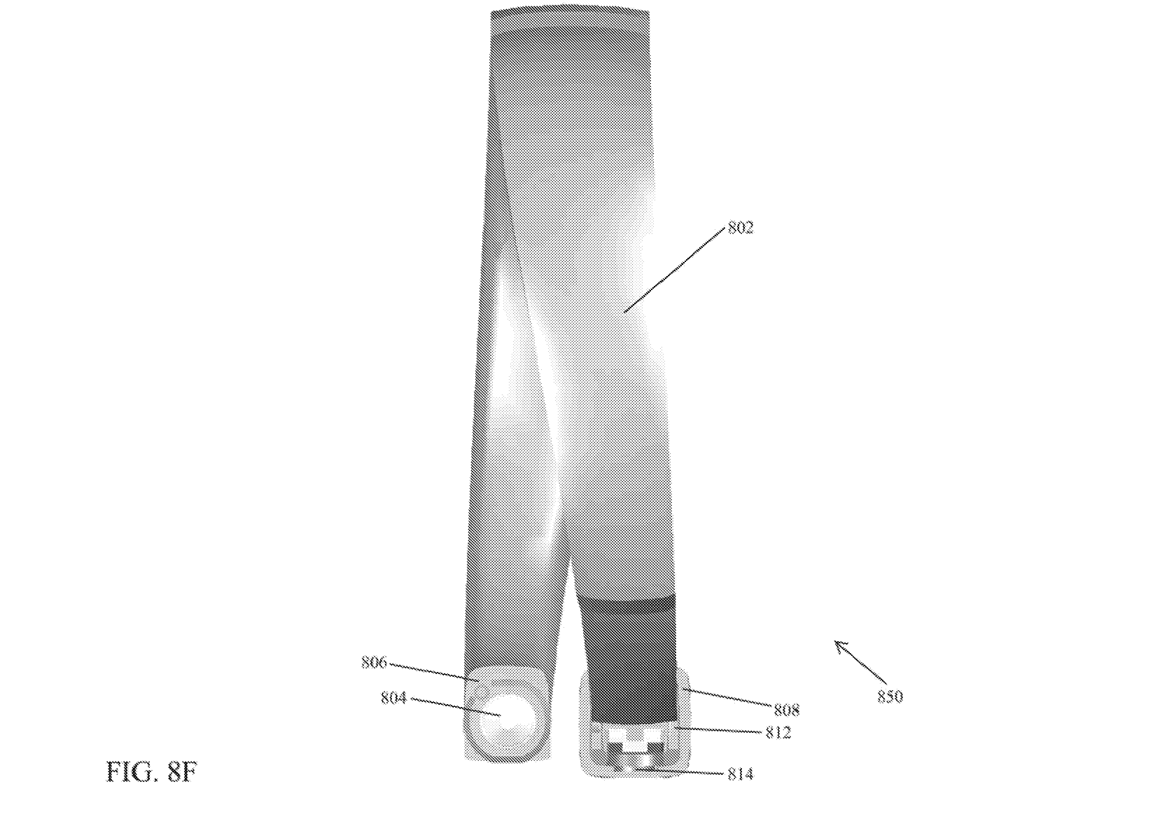

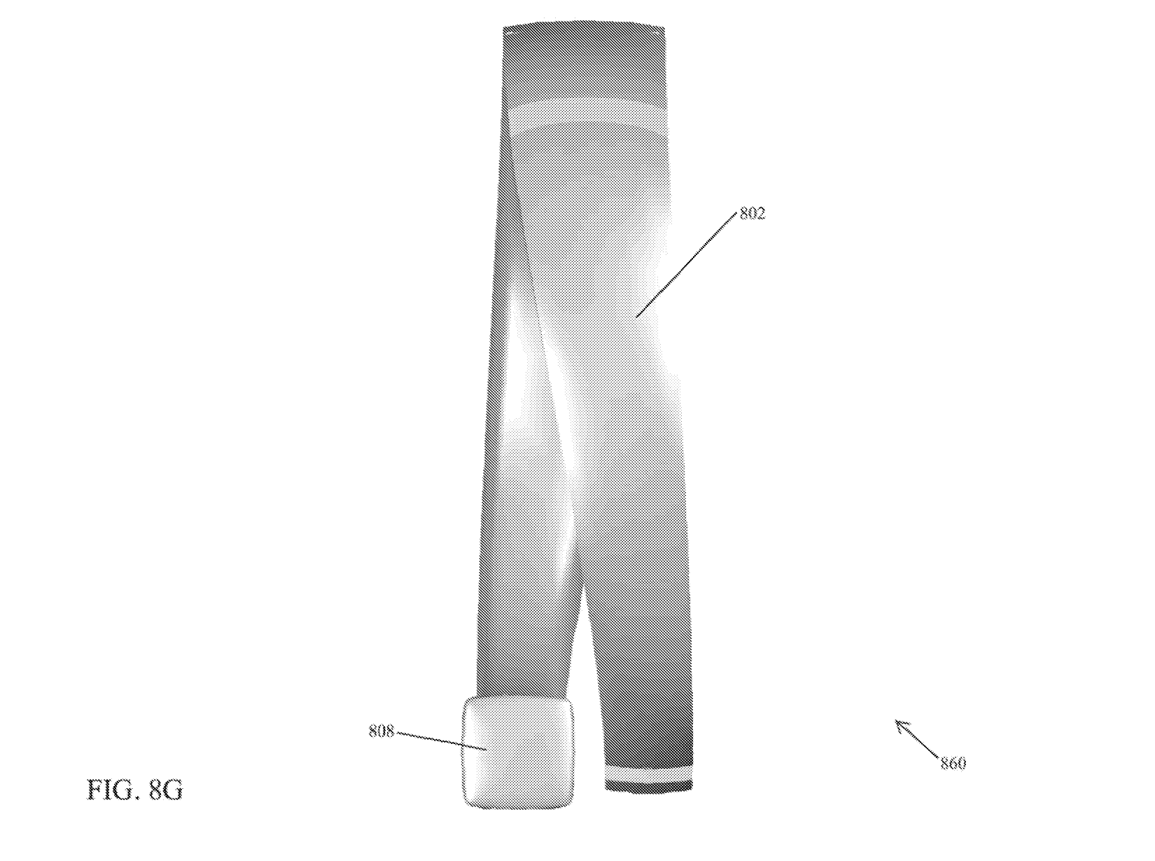

FIG. 8A illustrates a perspective of an exemplary data-capable strapband having a first molding. Here, an alternative band (i.e., band 800) includes molding 802, analog audio TRRS-type plug (hereafter “plug”) 804, plug housing 806, button 808, framework 810, control housing 812, and indicator light 814. In some examples, a first protective overmolding (i.e., molding 802) has been applied over band 700 (FIG. 7) and the above-described elements (e.g., covering 704, flexible circuit 706, covering 708, motor 710, coverings 714-724, plug 726, accessory 728, control housing 734, control 736, and flexible circuit 738) leaving some elements partially exposed (e.g., plug 804, plug housing 806, button 808, framework 810, control housing 812, and indicator light 814). However, internal PCBAs, flexible connectors, circuitry, and other sensitive elements have been protectively covered with a first or inner molding that can be configured to further protect band 800 from subsequent moldings formed over band 800 using the above-described techniques. In other examples, the type, configuration, location, shape, design, layout, or other aspects of band 800 may be varied and are not limited to those shown and described. For example, TRRS plug 804 may be removed if a wireless communication facility is instead attached to framework 810, thus having a transceiver, logic, and antenna instead being protected by molding 802. As another example, button 808 may be removed and replaced by another control mechanism (e.g., an accelerometer that provides motion data to a processor that, using firmware and/or an application, can identify and resolve different types of motion that band 800 is undergoing), thus enabling molding 802 to be extended more fully, if not completely, over band 800. In other examples, the number, type, function, configuration, ornamental appearance, or other aspects shown may be varied without limitation.

FIG. 8B illustrates a side view of an exemplary data-capable strapband. Here, band 820 includes molding 802, plug 804, plug housing 806, button 808, control housing 812, and indicator lights 814 and 822. In other examples, the number, type, function, configuration, ornamental appearance, or other aspects shown may be varied without limitation.

FIG. 8C illustrates another side view of an exemplary data-capable strapband. Here, band 825 includes molding 802, plug 804, button 808, framework 810, control housing 812, and indicator lights 814 and 822. The view shown is an opposite view of that presented in FIG. 8B. In other examples, the number, type, function, configuration, ornamental appearance, or other aspects shown may be varied without limitation.

FIG. 8D illustrates a top view of an exemplary data-capable strapband. Here, band 830 includes molding 802, plug 804, plug housing 806, button 808, control housing 812, and indicator lights 814 and 822. In other examples, the number, type, function, configuration, ornamental appearance, or other aspects shown may be varied without limitation.

FIG. 8E illustrates a bottom view of an exemplary data-capable strapband. Here, band 840 includes molding 802, plug 804, plug housing 806, button 808, control housing 812, and indicator lights 814 and 822. In other examples, the number, type, function, configuration, ornamental appearance, or other aspects shown may be varied without limitation.

FIG. 8F illustrates a front view of an exemplary data-capable strapband. Here, band 850 includes molding 802, plug 804, plug housing 806, button 808, control housing 812, and indicator light 814. In other examples, the number, type, function, configuration, ornamental appearance, or other aspects shown may be varied without limitation.

FIG. 8G illustrates a rear view of an exemplary data-capable strapband. Here, band 860 includes molding 802 and button 808. In other examples, the number, type, function, configuration, ornamental appearance, or other aspects shown may be varied without limitation.

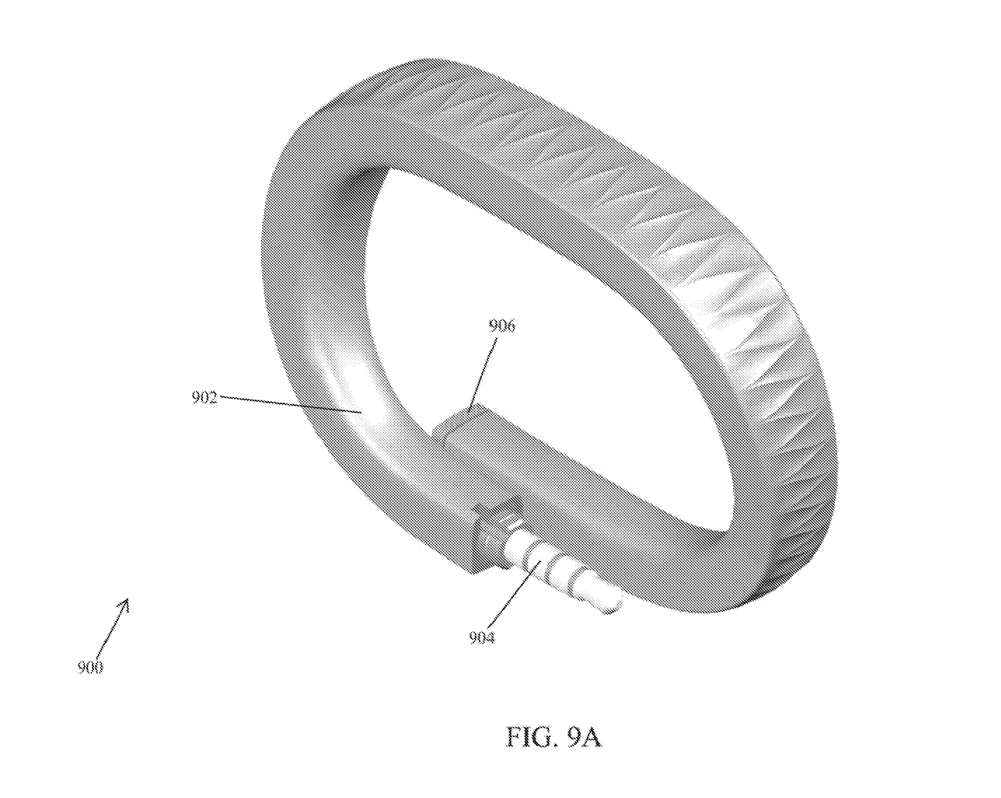

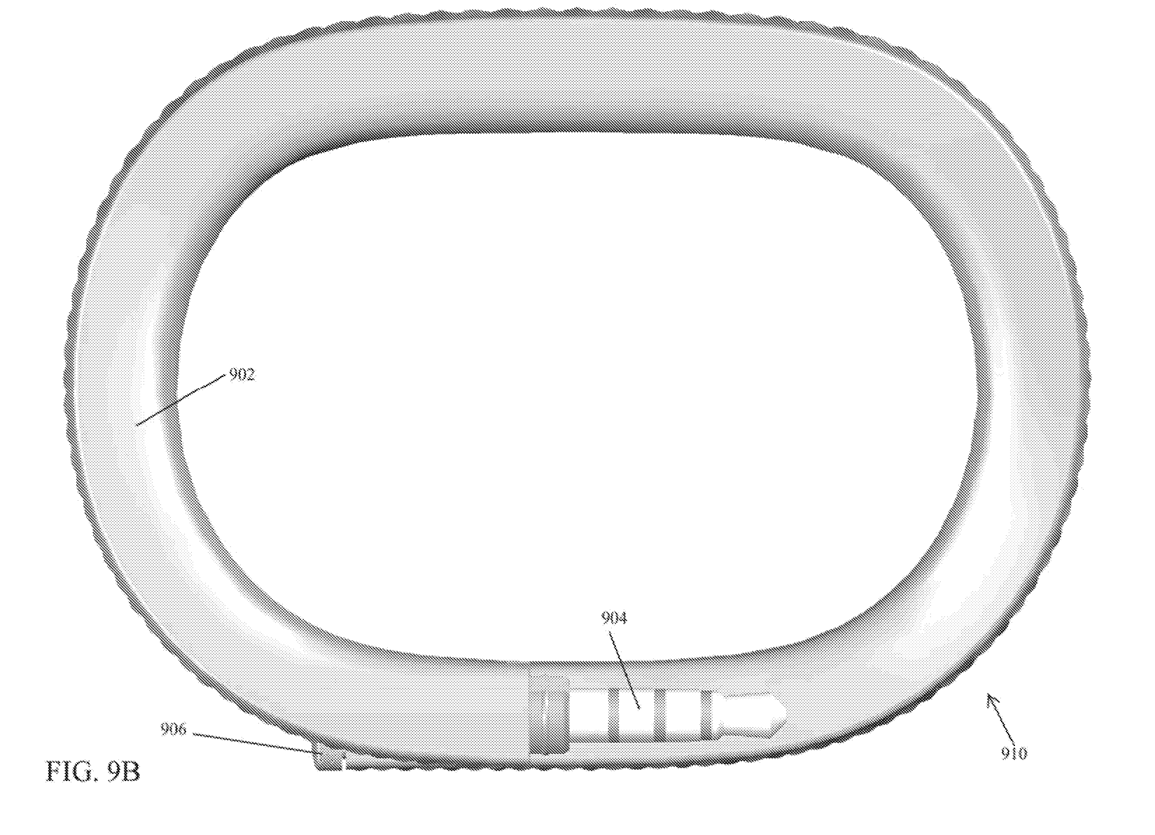

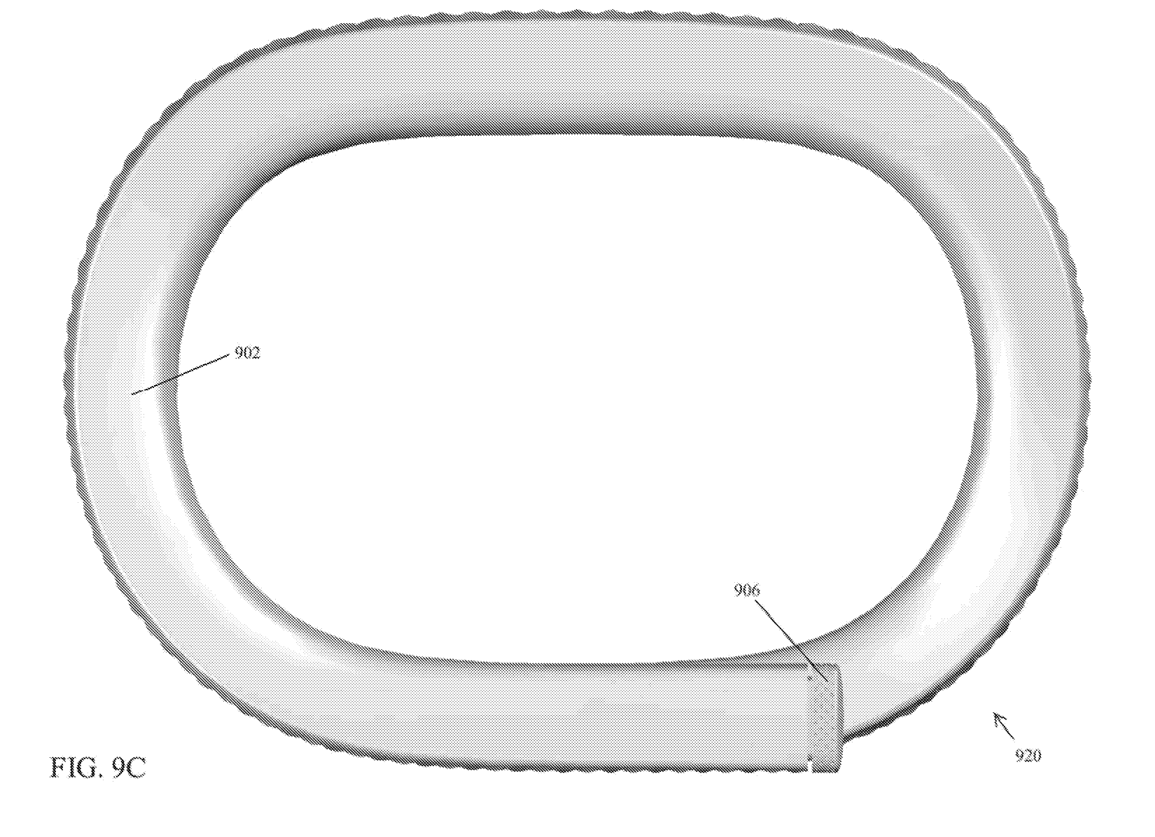

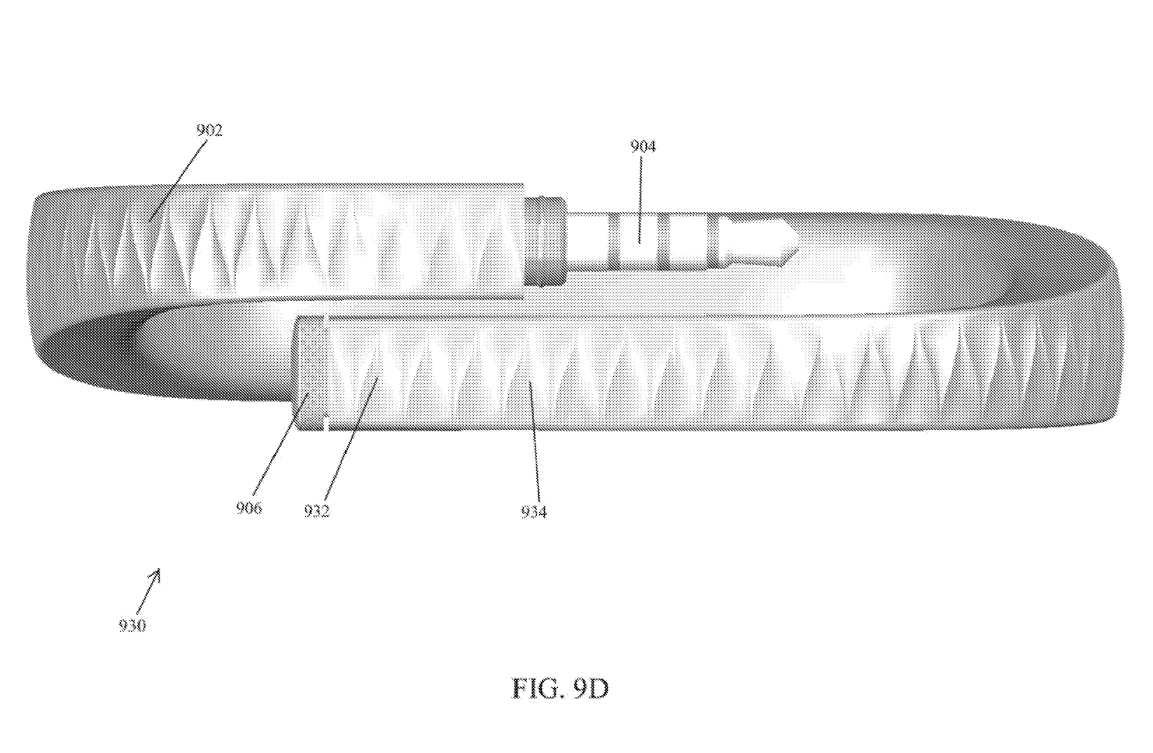

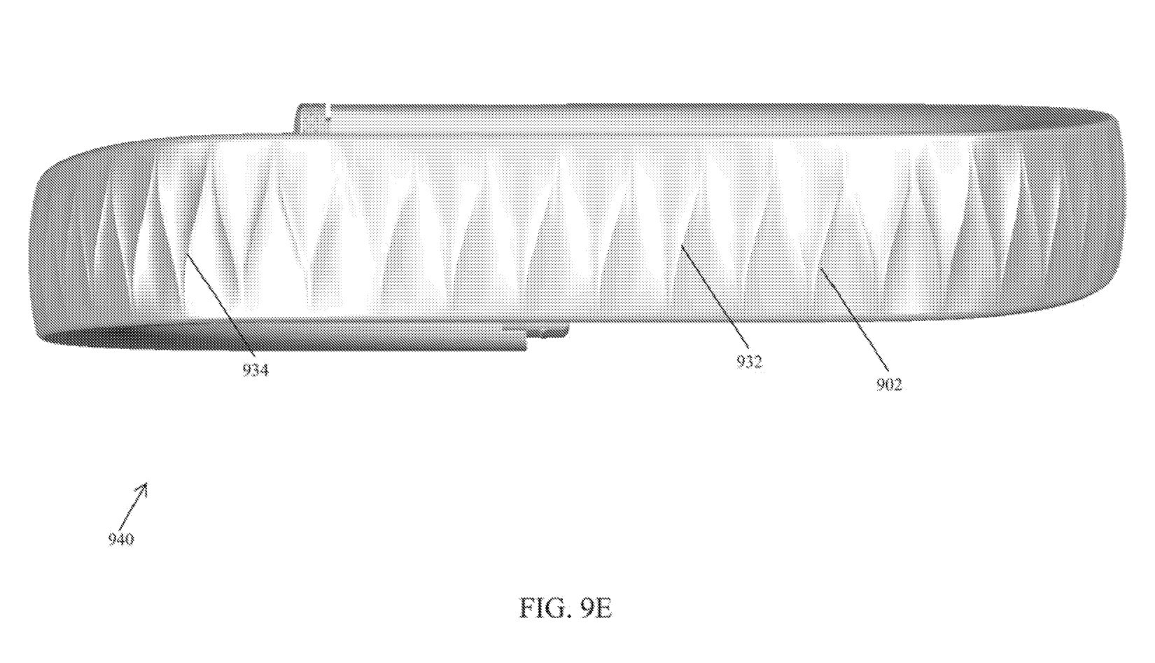

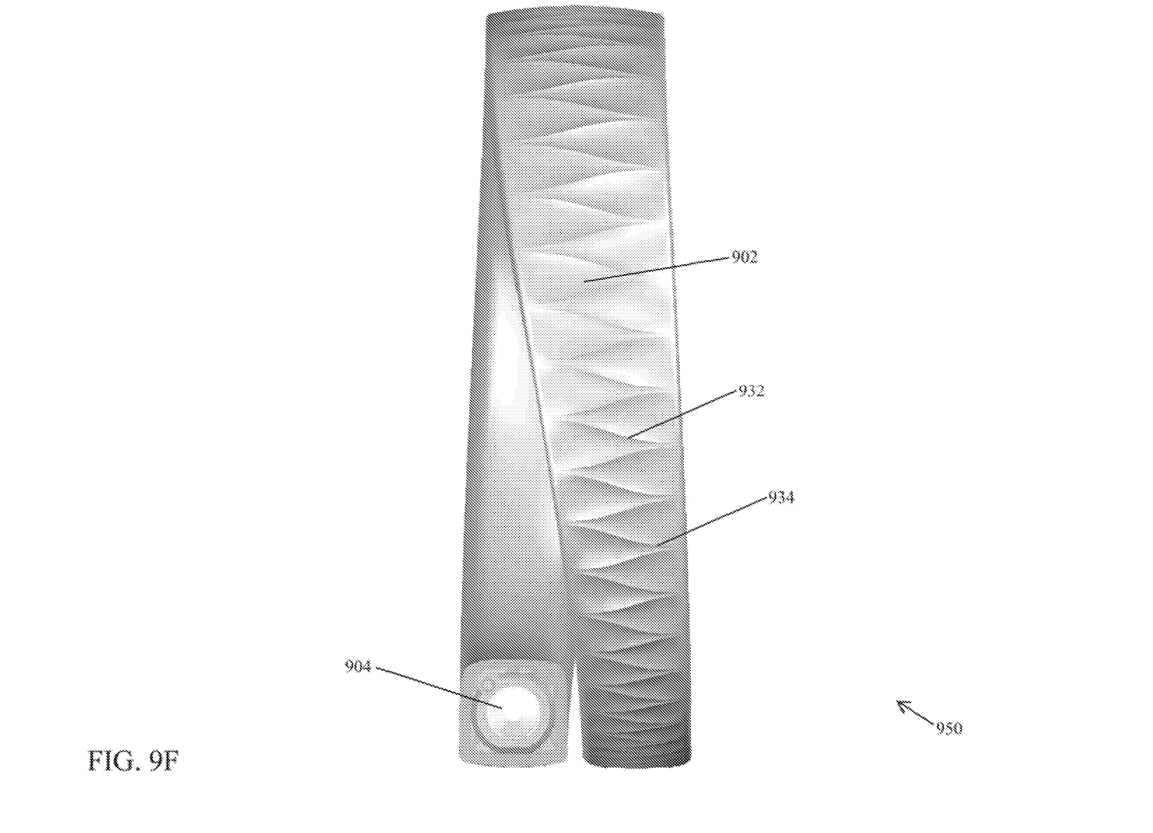

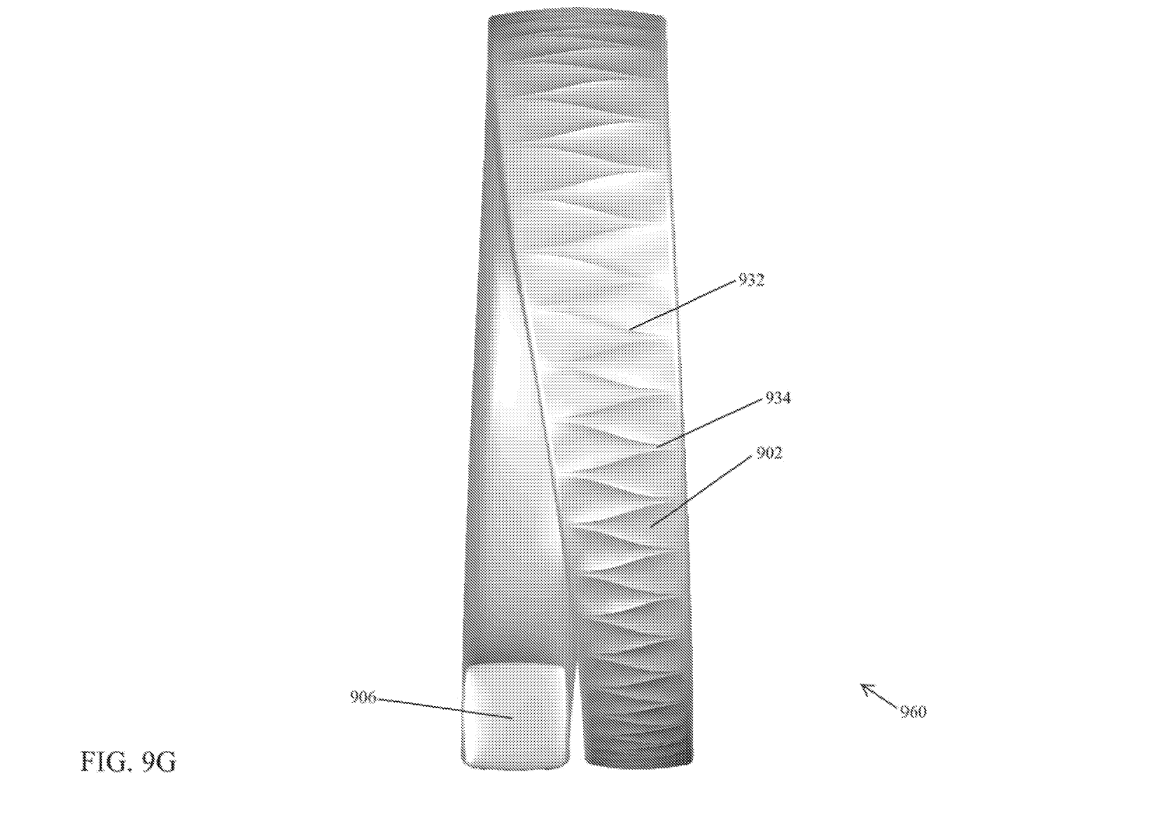

FIG. 9A illustrates a perspective view of an exemplary data-capable strapband having a second molding. Here, band 900 includes molding 902, plug 904, and button 906. As shown another overmolding or protective material has been formed by injection molding, for example, molding 902 over band 900. As another molding or covering layer, molding 902 may also be configured to receive surface designs, raised textures, or patterns, which may be used to add to the commercial appeal of band 900. In some examples, band 900 may be illustrative of a finished data-capable strapband (i.e., band 700 (FIG. 7), 800 (FIG. 8) or 900) that may be configured to provide a wide range of electrical, electronic, mechanical, structural, photonic, or other capabilities.

Here, band 900 may be configured to perform data communication with one or more other data-capable devices (e.g., other bands, computers, networked computers, clients, servers, peers, and the like) using wired or wireless features. For example, plug 900 may be used, in connection with firmware and software that allow for the transmission of audio tones to send or receive encoded data, which may be performed using a variety of encoded waveforms and protocols, without limitation. In other examples, plug 904 may be removed and instead replaced with a wireless communication facility that is protected by molding 902. If using a wireless communication facility and protocol, band 900 may communicate with other data-capable devices such as cell phones, smart phones, computers (e.g., desktop, laptop, notebook, tablet, and the like), computing networks and clouds, and other types of data-capable devices, without limitation. In still other examples, band 900 and the elements described above in connection with FIGS. 1-9, may be varied in type, configuration, function, structure, or other aspects, without limitation to any of the examples shown and described.

FIG. 9B illustrates a side view of an exemplary data-capable strapband. Here, band 910 includes molding 902, plug 904, and button 906. In other examples, the number, type, function, configuration, ornamental appearance, or other aspects shown may be varied without limitation.

FIG. 9C illustrates another side view of an exemplary data-capable strapband. Here, band 920 includes molding 902 and button 906. In other examples, the number, type, function, configuration, ornamental appearance, or other aspects shown may be varied without limitation.

FIG. 9D illustrates a top view of an exemplary data-capable strapband. Here, band 930 includes molding 902, plug 904, button 906, and textures 932-934. In some examples, textures 932-934 may be applied to the external surface of molding 902. As an example, textured surfaces may be molded into the exterior surface of molding 902 to aid with handling or to provide ornamental or aesthetic designs. The type, shape, and repetitive nature of textures 932-934 are not limiting and designs may be either two or three-dimensional relative to the planar surface of molding 902. In other examples, the number, type, function, configuration, ornamental appearance, or other aspects shown may be varied without limitation.

FIG. 9E illustrates a bottom view of an exemplary data-capable strapband. Here, band 940 includes molding 902 and textures 932-934, as described above. In other examples, the number, type, function, configuration, ornamental appearance, or other aspects shown may be varied without limitation.

FIG. 9F illustrates a front view of an exemplary data-capable strapband. Here, band 950 includes molding 902, plug 904, and textures 932-934. In other examples, the number, type, function, configuration, ornamental appearance, or other aspects shown may be varied without limitation.

FIG. 9G illustrates a rear view of an exemplary data-capable strapband. Here, band 960 includes molding 902, button 906, and textures 932-934. In other examples, the number, type, function, configuration, ornamental appearance, or other aspects shown may be varied without limitation.

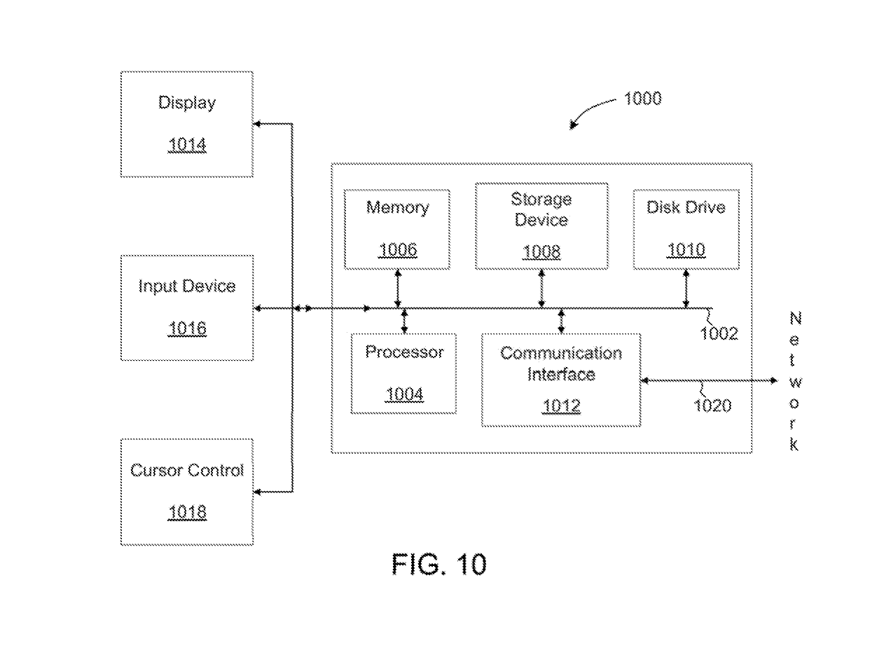

FIG. 10 illustrates an exemplary computer system suitable for use with a data-capable strapband. In some examples, computer system 1000 may be used to implement computer programs, applications, methods, processes, or other software to perform the above-described techniques. Computer system 1000 includes a bus 1002 or other communication mechanism for communicating information, which interconnects subsystems and devices, such as processor 1004, system memory 1006 (e.g., RAM), storage device 1008 (e.g., ROM), disk drive 1010 (e.g., magnetic or optical), communication interface 1012 (e.g., modem or Ethernet card), display 1014 (e.g., CRT or LCD), input device 1016 (e.g., keyboard), and cursor control 1018 (e.g., mouse or trackball).

According to some examples, computer system 1000 performs specific operations by processor 1004 executing one or more sequences of one or more instructions stored in system memory 1006. Such instructions may be read into system memory 1006 from another computer readable medium, such as static storage device 1008 or disk drive 1010. In some examples, hard-wired circuitry may be used in place of or in combination with software instructions for implementation.

The term “computer readable medium” refers to any tangible medium that participates in providing instructions to processor 1004 for execution. Such a medium may take many forms, including but not limited to, non-volatile media and volatile media. Non-volatile media includes, for example, optical or magnetic disks, such as disk drive 1010. Volatile media includes dynamic memory, such as system memory 1006.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1002 for transmitting a computer data signal.

In some examples, execution of the sequences of instructions may be performed by a single computer system 1000. According to some examples, two or more computer systems 1000 coupled by communication link 1020 (e.g., LAN, PSTN, or wireless network) may perform the sequence of instructions in coordination with one another. Computer system 1000 may transmit and receive messages, data, and instructions, including program, i.e., application code, through communication link 1020 and communication interface 1012. Received program code may be executed by processor 1004 as it is received, and/or stored in disk drive 1010, or other non-volatile storage for later execution.

FIG. 11 depicts a variety of inputs in a specific example of a strapband, such as a data-capable strapband, according to various embodiments. In diagram 1100, strapband 1102 can include one or more of the following: a switch 1104, a display I/O 1120, and a multi-pole or multi-position switch 1101. Switch 1104 can rotate in direction 1107 to select a mode, or switch 1104 can be a push button operable by pushing in direction 1105, whereby subsequent pressing of the button cycles through different modes of operation. Or, different sequences of short and long durations during which the button is activated. Display I/O 1120 can be a touch-sensitive graphical user interface. The multi-pole switch 1101, in some examples, can be a four-position switch, each position being associated with a mode (e.g., a sleep mode, an active mode, a normal mode, etc.). Additionally, commands can be entered via graphical user interface 1112 via wireless (or wired) communication device 1110. Further, any number of visual outputs (e.g., LEDs as indicator lights), audio outputs, and/or mechanical (e.g., vibration) outputs can be implemented to inform the user of an event, a mode, or any other status of interest relating to the functionality of the strapband.

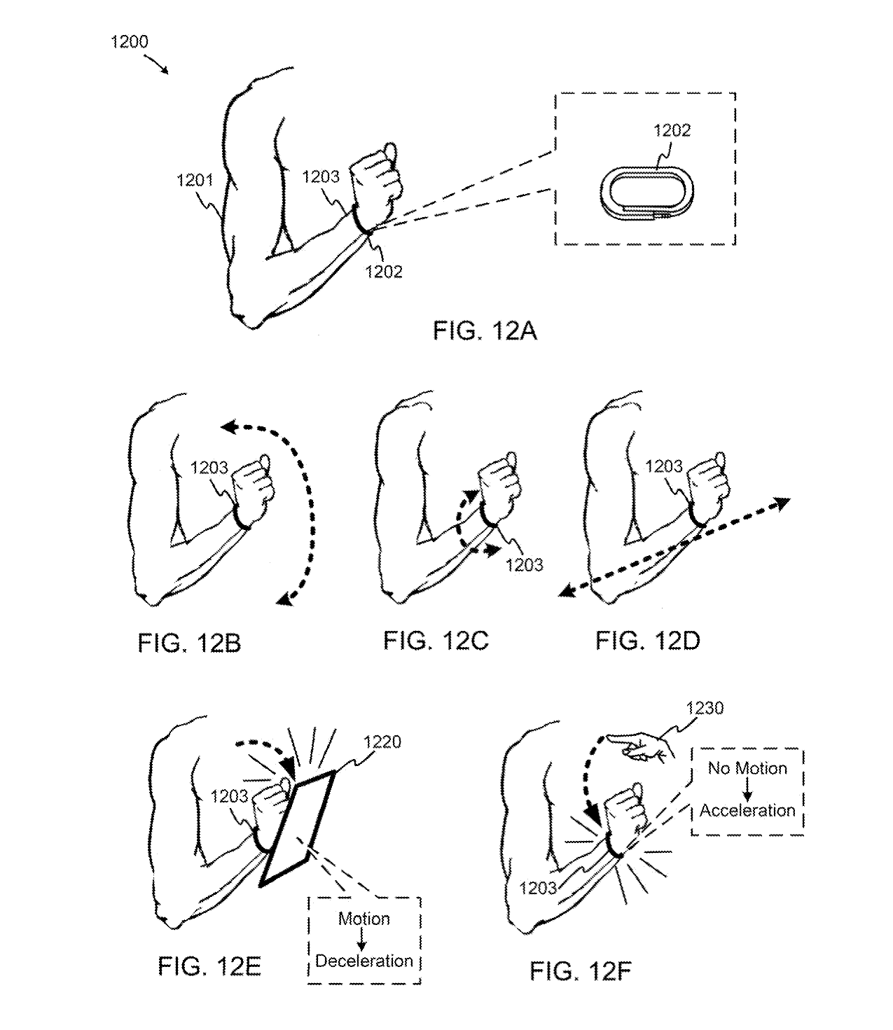

FIGS. 12A to 12F depict a variety of motion signatures as input into a strapband, such as a data-capable strapband, according to various embodiments. In FIG. 12A, diagram 1200 depicts a user's arm (e.g., as a locomotive member or appendage) IS with a strapband 1202 attached to user wrist 1203. Strapband 1202 can envelop or substantially surround user wrist 1203 as well. FIGS. 12B to 12D illustrate different “motion signatures” defined by various ranges of motion and/or motion patterns (as well as number of motions), whereby each of the motion signatures identifies a mode of operation. FIG. 12B depicts up-and-down motion, FIG. 12C depicts rotation about the wrist, and FIG. 12D depicts side-to-side motion. FIG. 12E depicts an ability detect a change in mode as a function of the motion and deceleration (e.g., when a user claps hands or makes contact with a surface 1220 to get strapband to change modes), whereas, FIG. 12F depicts an ability to detect “no motion” initially and experience an abrupt acceleration of the strapband (e.g., user taps strapband with finger 1230 to change modes). Note that motion signatures can be motion patterns that are predetermined, with the user selecting or linking a specific motion signature to invoke a specific mode. Note, too, a user can define unique motion signatures. In some embodiments, any number of detect motions can be used to define a motion signature. Thus, different numbers of the same motion can activate different modes. For example, two up-and-down motions in FIG. 12B can activate one mode, whereas four up-and-down motions can activate another mode. Further, any combination of motions (e.g., two up-and-down motions of FIG. 12B and two taps of FIG. 12E) can be used as an input, regardless whether a mode of operation or otherwise.

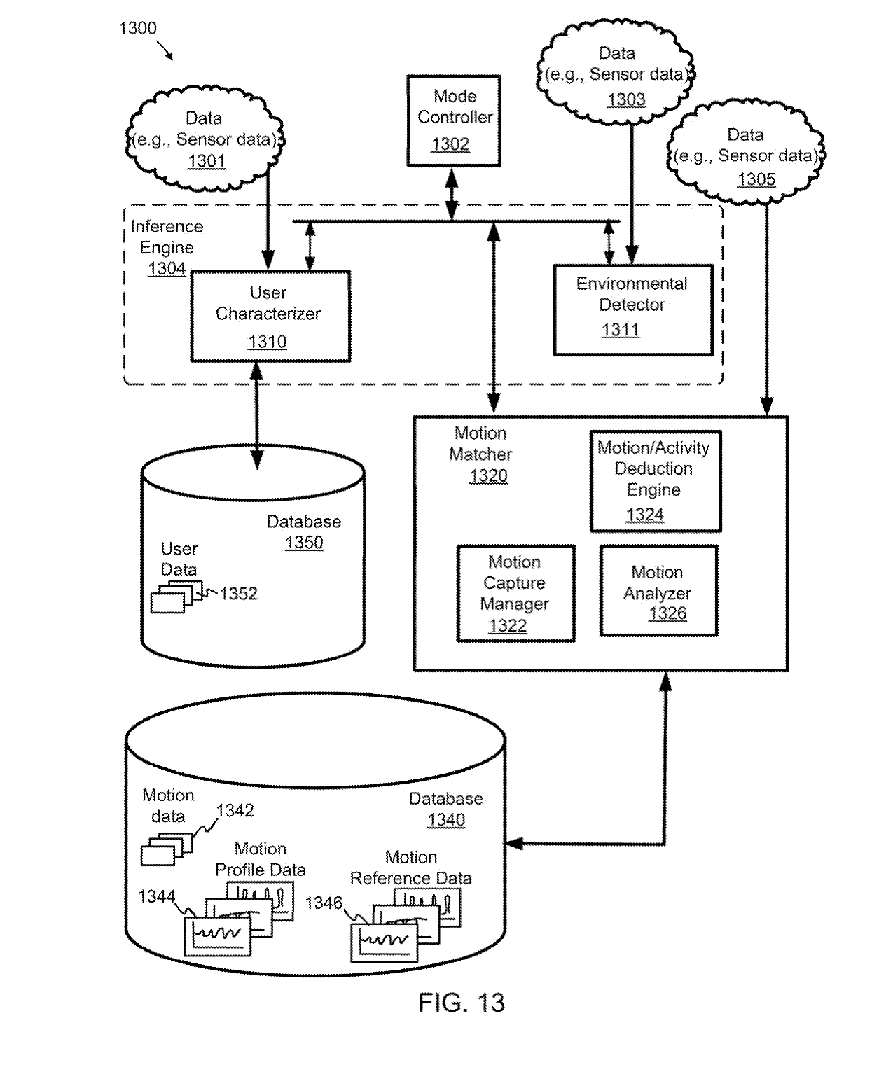

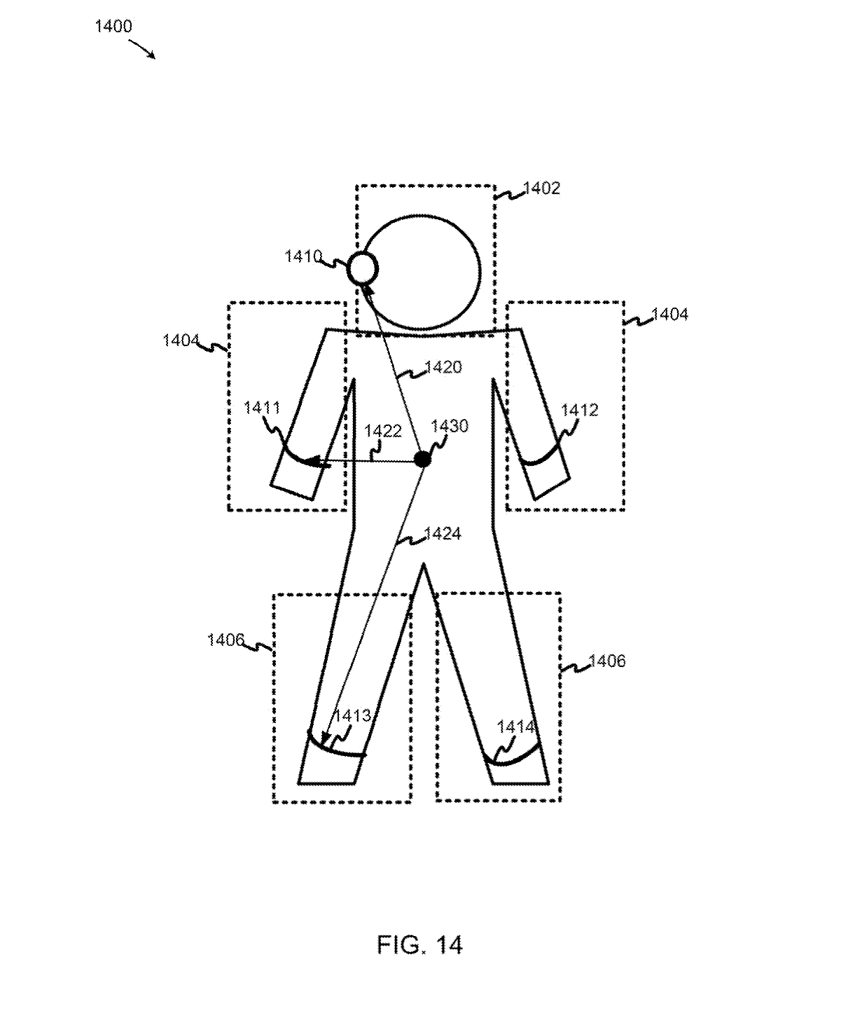

FIG. 13 depicts an inference engine of a strapband configured to detect an activity and/or a mode based on monitored motion, according to various embodiments. In some embodiments, inference engine 1304 of a strapband can be configured to detect an activity or mode, or a state of a strapband, as a function of at least data derived from one or more sources of data, such as any number of sensors. Examples of data obtained by the sensors include, but are not limited to, data describing motion, location, user characteristics (e.g., heart rate, body temperature, etc.), environmental characteristics (e.g., time, degree of ambient light, altitude, magnetic flux (e.g., magnetic field of the earth), or any other source of magnetic flux), GPS-generated position data, proximity to other strapband wearers, etc.), and data derived or sensed by any source of relevant information. Further, inference engine 1304 is configured to analyze sets of data from a variety of inputs and sources of information to identify an activity, mode and/or state of a strapband. In one example, a set of sensor data can include GPS-derived data, data representing magnetic flux, data representing rotation (e.g., as derived by a gyroscope), and any other data that can be relevant to inference engine 1304 in its operation. The inference engine can use positional data along with motion-related information to identify an activity or mode, among other purposes.

According to some embodiments, inference engine 1304 can be configured to analyze real-time sensor data, such as user-related data 1301 derived in real-time from sensors and/or environmental-related data 1303 derived in real-time from sensors. In particular, inference engine 1304 can compare any of the data derived in real-time (or from storage) against other types of data (regardless of whether the data is real-time or archived). The data can originate from different sensors, and can obtained in real-time or from memory as user data 1352. Therefore, inference engine 1304 can be configured to compare data (or sets of data) against each other, thereby matching sensor data, as well as other data, to determine an activity or mode.

Diagram 1300 depicts an example of an inference engine 1304 that is configured to determine an activity in which the user is engaged, as a function of motion and, in some embodiments, as a function of sensor data, such as user-related data 1301 derived from sensors and/or environmental-related data 1303 derived from sensors. Examples of activities that inference engine 1304 evaluates include sitting, sleeping, working, running, walking, playing soccer or baseball, swimming, resting, socializing, touring, visiting various locations, shopping at a store, and the like. These activities are associated with different motions of the user, and, in particular, different motions of one or more locomotive members (e.g., motion of a user's arm or wrist) that are inherent in the different activities. For example, a user's wrist motion during running is more “pendulum-like” in it motion pattern, whereas, the wrist motion during swimming (e.g., freestyle strokes) is more “circular-like” in its motion pattern. Diagram 1300 also depicts a motion matcher 1320, which is configured to detect and analyze motion to determine the activity (or the most probable activity) in which the user is engaged. To further refine the determination of the activity, inference engine 1304 includes a user characterizer 1310 and an environmental detector 1311 to detect sensor data for purposes of comparing subsets of sensor data (e.g., one or more types of data) against other subsets of data. Upon determining a match between sensor data, inference engine 1304 can use the matched sensor data, as well as motion-related data, to identify a specific activity or mode. User characterizer 1310 is configured to accept user-related data 1301 from relevant sensors. Examples of user-related data 1301 include heart rate, body temperature, or any other personally-related information with which inference engine 1304 can determine, for example, whether a user is sleeping or not. Further, environmental detector 1311 is configured to accept environmental-related data 1303 from relevant sensors. Examples of environmental-related data 1303 include time, ambient temperature, degree of brightness (e.g., whether in the dark or in sunlight), location data (e.g., GPS data, or derived from wireless networks), or any other environmental-related information with which inference engine 1304 can determine whether a user is engaged in a particular activity.

A strapband can operate in different modes of operation. One mode of operation is an “active mode.” Active mode can be associated with activities that involve relatively high degrees of motion at relatively high rates of change. Thus, a strapband enters the active mode to sufficiently capture and monitor data with such activities, with power consumption as being less critical. In this mode, a controller, such as mode controller 1302, operates at a higher sample rate to capture the motion of the strapband at, for example, higher rates of speed. Certain safety or health-related monitoring can be implemented in active mode, or, in response to engaging in a specific activity. For example, a controller of strapband can monitor a user's heart rate against normal and abnormal heart rates to alert the user to any issues during, for example, a strenuous activity. In some embodiments, strapband can be configured as set forth in FIG. 5B and user characterizer 1310 can process user-related information from sensors described in relation FIG. 5B. Another mode of operation is a “sleep mode.” Sleep mode can be associated with activities that involve relatively low degrees of motion at relatively low rates of change. Thus, a strapband enters the sleep mode to sufficiently capture and monitor data with such activities, while preserving power. In some embodiments, strapband can be configured as set forth in FIG. 5C and user characterizer 1310 can process user-related information from sensors described in relation FIG. 5C. Yet another mode is “normal mode,” in which the strapband operates in accordance with typical user activities, such as during work, travel, movement around the house, bathing, etc. A strapband can operate in any number different modes, including a health monitoring mode, which can implement, for example, the features set forth in FIG. 5D. Another mode of operation is a “social mode” of operation in which the user interacts with other users of similar strapbands or communication devices, and, thus, a strapband can implement, for example, the features set forth in FIG. 5E. Any of these modes can be entered or exited either explicitly or implicitly.

Diagram 1300 also depicts a motion matcher 1320, which is configured to detect and analyze motion to determine the activity (or the most probable activity) in which the user is engaged. In various embodiments, motion matcher 1320 can form part of inference engine 1304 (not shown), or can have a structure and/or function separate therefrom (as shown). Regardless, the structures and/or functions of inference engine 1304, including user characterizer 1310 and an environmental detector 1311, and motion matcher 1320 cooperate to determine an activity in which the user is engaged and transmit data indicating the activity (and other related information) to a controller (e.g., a mode controller 1302) that is configured to control operation of a mode, such as an “active mode,” of the strapband.

Motion matcher 1320 of FIG. 13 includes a motion/activity deduction engine 1324, a motion capture manager 1322 and a motion analyzer 1326. Motion matcher 1320 can receive motion-related data 1303 from relevant sensors, including those sensors that relate to space or position and to time. Examples of such sensors include accelerometers, motion detectors, velocimeters, altimeters, barometers, etc. Motion capture manager 1322 is configured to capture portions of motion, and to aggregate those portions of motion to form an aggregated motion pattern or profile. Further, motion capture manager 1322 is configured to store motion patterns as profiles 1344 in database 1340 for real-time or future analysis. Motion profiles 1344 include sets of data relating to instances of motion or aggregated portions of motion (e.g., as a function of time and space, such as expressed in X, Y, Z coordinate systems).

For example, motion capture manager 1322 can be configured to capture motion relating to the activity of walking and motion relating to running, each motion being associated with a specific profile 1344. To illustrate, consider that motion profiles 1344 of walking and running share some portions of motion in common. For example, the user's wrist motion during running and walking share a “pendulum-like” pattern over time, but differ in sampled positions of the strapband. During walking, the wrist and strapband is generally at waist-level as the user walks with arms relaxed (e.g., swinging of the arms during walking can result in a longer arc-like motion pattern over distance and time), whereas during running, a user typically raises the wrists and changes the orientation of the strapband (e.g., swinging of the arms during running can result in a shorter arc-like motion pattern). Motion/activity deduction engine 1324 is configured to access profiles 1344 and deduce, for example, in real-time whether the activity is walking or running.

Motion/activity deduction engine 1324 is configured to analyze a portion of motion and deduce the activity (e.g., as an aggregate of the portions of motion) in which the user is engaged and provide that information to the inference engine 1304, which, in turn, compares user characteristics and environmental characteristics against the deduced activity to confirm or reject the determination. For example, if motion/activity deduction engine 1324 deduces that monitored motion indicates that the user is sleeping, then the heart rate of the user, as a user characteristic, can be used to compare against thresholds in user data 1352 of database 1350 to confirm that the user's heart rate is consistent with a sleeping user. User data 1352 can also include past location data, whereby historic location data can be used to determine whether a location is frequented by a user (e.g., as a means of identifying the user). Further, inference engine 1304 can evaluate environmental characteristics, such as whether there is ambient light (e.g., darkness implies conditions for resting), the time of day (e.g., a person's sleeping times typically can be between 12 midnight and 6 am), or other related information.

In operation, motion/activity deduction engine 1324 can be configured to store motion-related data to form motion profiles 1344 in real-time (or near real-time). In some embodiments, the motion-related data can be compared against motion reference data 1346 to determine “a match” of motions. Motion reference data 1346, which includes reference motion profiles and patterns, can be derived by motion data captured for the user during previous activities, whereby the previous activities and motion thereof serve as a reference against which to compare. Or, motion reference data 1346 can include ideal or statistically-relevant motion patterns against which motion/activity deduction engine 1324 determines a match by determining which reference profile data 1346 “best fits” the real-time motion data. Motion/activity deduction engine 1324 can operate to determine a motion pattern, and, thus, determine an activity. Note that motion reference profile data 1346, in some embodiments, serves as a “motion fingerprint” for a user and can be unique and personal to a specific user. Therefore, motion reference profile data 1346 can be used by a controller to determine whether subsequent use of a strapband is by the authorized user or whether the current user's real-time motion data is a mismatch against motion reference profile data 1346. If there is mismatch, a controller can activate a security protocol responsive to the unauthorized use to preserve information or generate an alert to be communicated external to the strapband.

Motion analyzer 1326 is configured to analyze motion, for example, in real-time, among other things. For example, if the user is swinging a baseball bat or golf club (e.g., when the strapband is located on the wrist) or the user is kicking a soccer ball (e.g., when the strapband is located on the ankle), motion analyzer 1326 evaluates the captured motion to detect, for example, a deceleration in motion (e.g., as a motion-centric event), which can be indicative of an impulse event, such as striking an object, like a golf ball. Motion-related characteristics, such as space and time, as well as other environment and user characteristics can be captured relating to the motion-centric event. A motion-centric event, for example, is an event that can relate to changes in position during motion, as well as changes in time or velocity. In some embodiments, inference engine 1304 stores user characteristic data and environmental data in database 1350 as user data 1352 for archival purposes, reporting purposes, or any other purpose. Similarly inference engine 1304 and/or motion matcher 1320 can store motion-related data as motion data 1342 for real-time and/or future use. According to some embodiments, stored data can be accessed by a user or any entity (e.g., a third party) to adjust the data of databases 1340 and 1350 to, for example, optimize motion profile data or sensor data to ensure more accurate results. A user can access motion profile data in database 1350. Or, a user can adjust the functionality of inference engine 1304 to ensure more accurate or precise determinations. For example, if inference engine 1304 detects a user's walking motion as a running motion, the user can modify the behavior of the logic in the strapband to increase the accuracy and optimize the operation of the strapband.

FIG. 14 depicts a representative implementation of one or more strapbands and equivalent devices, as wearable devices, to form unique motion profiles, according to various embodiments. In diagram 1400, strapbands and an equivalent device are disposed on locomotive members of the user, whereby the locomotive members facilitate motion relative to and about a center point 1430 (e.g., a reference point for a position, such as a center of mass). A headset 1410 is configured to communicate with strapbands 1411, 1412, 1413 and 1414 and is disposed on a body portion 1402 (e.g., the head), which is subject to motion relative to center point 1430. Strapbands 1411 and 1412 are disposed on locomotive portions 1404 of the user (e.g., the arms or wrists), whereas strapbands 1413 and 1414 are disposed on locomotive portion 1406 of the user (e.g., the legs or ankles). As shown, headset 1410 is disposed at distance 1420 from center point 1430, strapbands 1411 and 1412 are disposed at distance 1422 from center point 1430, and strapbands 1413 and 1414 are disposed at distance 1424 from center point 1430. A great number of users have different values of distances 1420, 1422, and 1424. Further, different wrist-to-elbow and elbow-to-shoulder lengths for different users affect the relative motion of strapbands 1411 and 1412 about center point 1430, and similarly, different hip-to-knee and knee-to-ankle lengths for different users affect the relative motion of strapbands 1413 and 1414 about center point 1430. Moreover, a great number of users have unique gaits and styles of motion. The above-described factors, as well as other factors, facilitate the determination of a unique motion profile for a user per activity (or in combination of a number of activities). The uniqueness of the motion patterns in which a user performs an activity enables the use of motion profile data to provide a “motion fingerprint.” A “motion fingerprint” is unique to a user and can be compared against detected motion profiles to determine, for example, whether a use of the strapband by a subsequent wearer is unauthorized. In some cases, unauthorized users do not typically share common motion profiles. Note that while four are shown, fewer than four can be used to establish a “motion fingerprint,” or more can be shown (e.g., a strapband can be disposed in a pocket or otherwise carried by the user). For example, a user can place a single strapbands at different portions of the body to capture motion patterns for those body parts in a serial fashion. Then, each of the motions patterns can be combined to form a “motion fingerprint.” In some cases, a single strapband 1411 is sufficient to establish a “motion fingerprint.” Note, too, that one or more of strapbands 1411, 1412, 1413 and 1414 can be configured to operate with multiple users, including non-human users, such as pets.

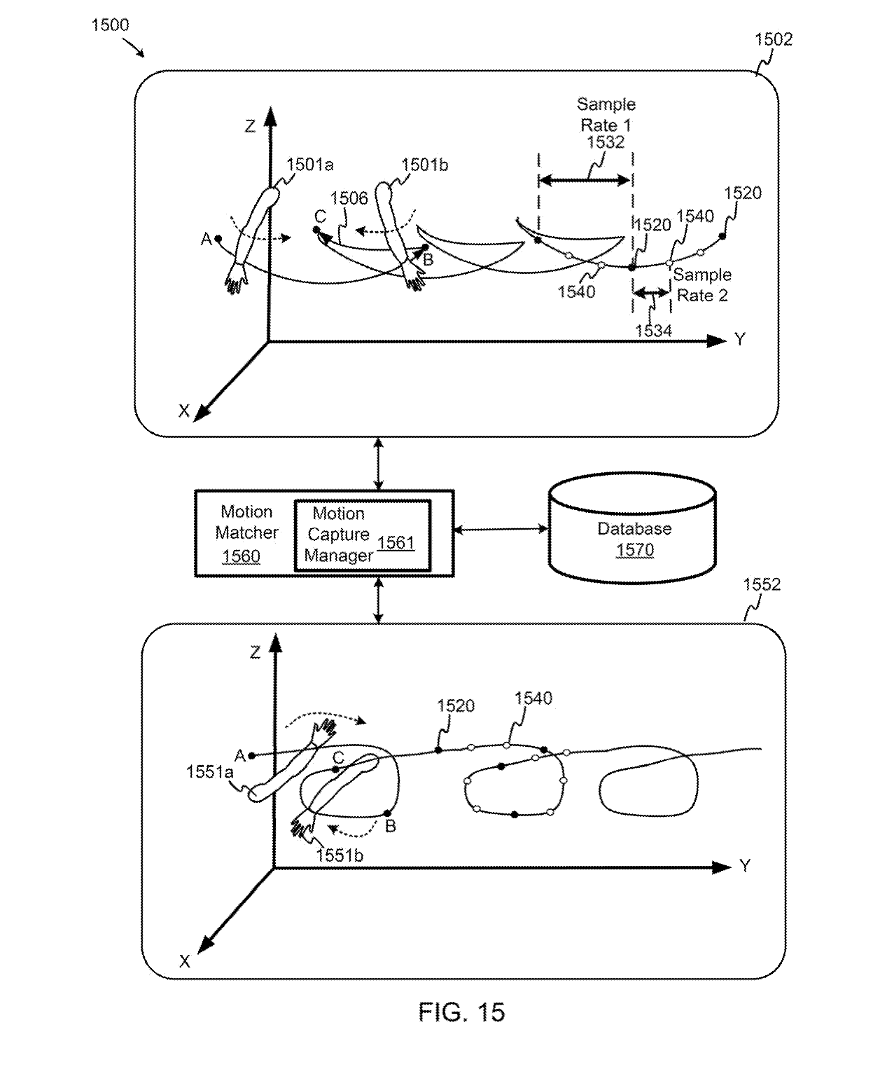

FIG. 15 depicts an example of a motion capture manager configured to capture motion and portions therefore, according to various embodiments. Diagram 1500 depicts an example of a motion matcher 1560 and/or a motion capture manager 1561, one or both of which are configured to capture motion of an activity or state of a user and generate one or more motion profiles, such as motion profile 1502 and motion profile 1552. Database 1570 is configured to store motion profiles 1502 and 1552. Note that motion profiles 1502 and 1552 are shown as graphical representation of motion data for purposes of discussion, and can be stored in any suitable data structure or arrangement. Note, too, that motion profiles 1502 and 1552 can represent real-time motion data with which a motion matcher 1560 uses to determine modes and activities.

To illustrate operation of motion capture manager 1561, consider that motion profile 1502 represents motion data captured for a running or walking activity. The data of motion profile 1502 indicates the user is traversing along the Y-axis with motions describable in X, Y, Z coordinates or any other coordinate system. The rate at which motion is captured along the Y-axis is based on the sampling rate and includes a time component. For a strapband disposed on a wrist of a user, motion capture manager 1561 captures portions of motion, such as repeated motion segments A-to-B and B-to-C. In particular, motion capture manager 1561 is configured to detect motion for an arm 1501a in the +Y direction from the beginning of the forward swinging arm (e.g., point A) to the end of the forward swinging arm (e.g., point B). Further, motion capture manager 1561 is configured to detect motion for arm 1501b in the −Y direction from the beginning of the backward swinging arm (e.g., point 13) to the end of the backward swinging arm (e.g., point C). Note that point C is at a greater distance along the Y-axis than point A as the center point or center mass of the user has advanced in the +Y direction. Motion capture manager 1561 continues to monitor and capture motion until, for example, motion capture manager 1561 detects no significant motion (i.e., below a threshold) or an activity or mode is ended.

Note that in some embodiments, a motion profile can be captured by motion capture manager 1561 in a “normal mode” of operation and sampled at a first sampling rate (“sample rate 1”) 1532 between samples of data 1520, which is a relatively slow sampling rate that is configured to operate with normal activities. Samples of data 1520 represent not only motion data (e.g., data regarding X, Y, and Z coordinates, time, accelerations, velocities, etc.), but can also represent or link to user related information captured at those sample times. Motion matcher 1560 analyzes the motion, and, if the motion relates to an activity associated with an “active mode,” motion matcher 1560 signals to the controller, such as a mode controller, to change modes (e.g., from normal to active mode). During active mode, the sampling rate increases to a second sampling rate (“sample rate 2”) 1534 between samples of data 1520 (e.g. as well as between a sample of data 1520 and a sample of data 1540). An increased sampling rate can facilitate, for example, a more accurate set of captured motion data. To illustrate the above, consider that a user is sitting or stretching prior to a work out. The user's activities likely are occurring in a normal mode of operation. But once motion data of profile 1502 is detected, a motion/activity deduction engine can deduce the activity of running, and then can infer the mode ought to be the active mode. The logic of the strapband then can place the strapband into the active mode. Therefore, the strapband can change modes of operation implicitly (i.e., explicit actions to change modes need not be necessary). In some cases, a mode controller can identify an activity as a “running” activity, and then invoke activity-specific functions, such as an indication (e.g., a vibratory indication) to the user every one-quarter mile or 15 minute duration during the activity.

FIG. 15 also depicts another motion profile 1552. Consider that motion profile 1552 represents motion data captured for swimming activity (e.g., using a freestyle stroke). Similar to profile 1502, the motion pattern data of motion profile 1552 indicates the user is traversing along the Y-axis. The rate at which motion is captured along the Y-axis is based on the sampling rate of samples 1520 and 1540, for example. For a strapband disposed on a wrist of a user, motion capture manager 1561 captures the portions of motion, such as motion segments A-to-B and B-to-C. In particular, motion capture manager 1561 is configured to detect motion for an arm 1551a in the +Y direction from the beginning of a forward arc (e.g., point A) to the end of the forward arc (e.g., point B). Further, motion capture manager 1561 is configured to detect motion for arm 1551b in the −Y direction from the beginning of reverse arc (e.g., point B) to the end of the reverse arc (e.g., point C). Motion capture manager 1561 continues to monitor and capture motion until, for example, motion capture manager 1561 detects no significant motion (i.e., below a threshold) or an activity or mode is ended.

In operation, a mode controller can determine that the motion data of profile 1552 is associated with an active mode, similar with the above-described running activity, and can place the strapband into the active mode, if it is not already in that mode. Further, motion matcher 1560 can analyze the motion pattern data of profile 1552 against, for example, the motion data of profile 1502 and conclude that the activity associated with the data being captured for profile 1552 does not relate to a running activity. Motion matcher 1560 then can analyze profile 1552 of the real-time generated motion data, and, if it determines a match with reference motion data for the activity of swimming, motion matcher 1560 can generate an indication that the user is performing “swimming” as an activity. Thus, the strapband and its logic can implicitly determine an activity that a user is performing (i.e., explicit actions to specify an activity need not be necessary). Therefore, a mode controller then can invoke swimming-specific functions, such as an application to generate an indication (e.g., a vibratory indication) to the user at completion of every lap, or can count a number of strokes. While not shown, motion matcher 1560 and/or a motion capture manager 1561 can be configured to implicitly determine modes of operation, such as a sleeping mode of operation (e.g., the mode controller, in part, can analyze motion patterns against a motion profile that includes sleep-related motion data. Motion matcher 1560 and/or a motion capture manager 1561 also can be configured to an activity out of a number of possible activities.

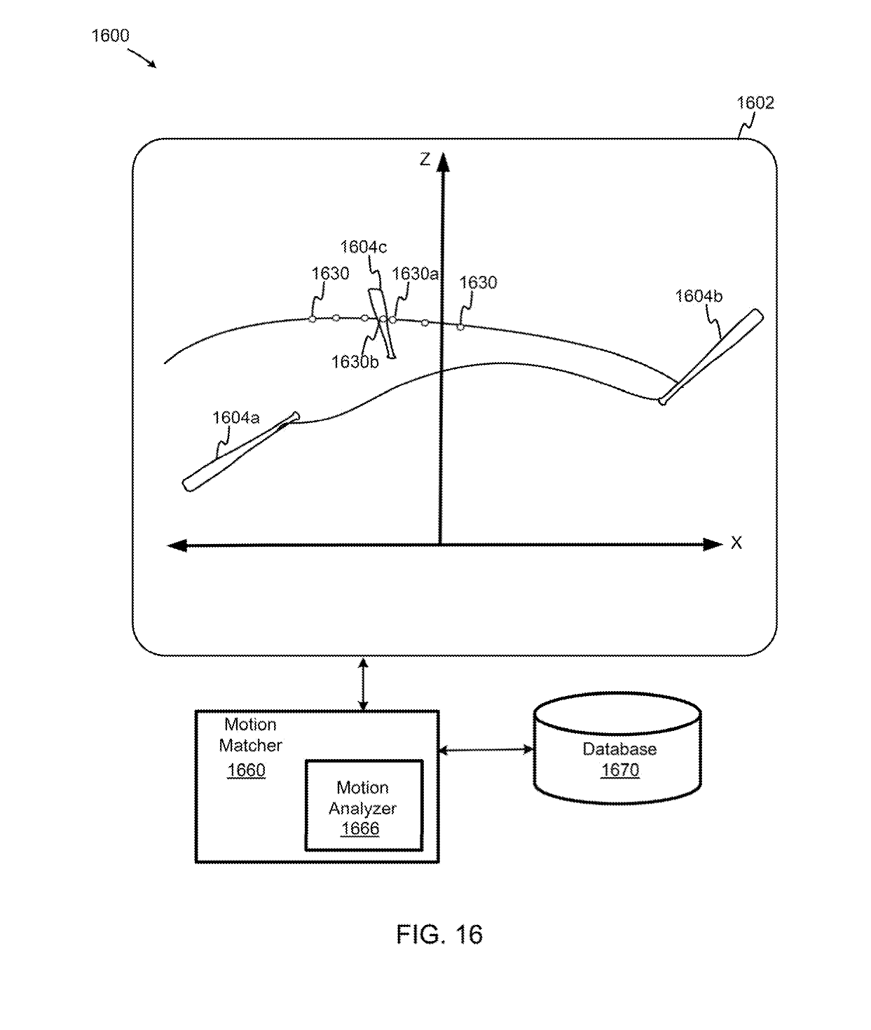

FIG. 16 depicts an example of a motion analyzer configured to evaluate motion-centric events, according to various embodiments. Diagram 1600 depicts an example of a motion matcher 1660 and/or a motion analyzer 1666 for capturing motion of an activity or state of a user and generating one or more motion profiles, such as a motion profile 1602. To illustrate, consider that motion profile 1602 represents motion data captured for an activity of swinging a baseball bat 1604. The motion pattern data of motion profile 1602 indicates the user begins the swing at position 1604a in the −Y direction. The user moves the strapband and the bat to position 1604b, and then swings the bat toward the −Y direction when contact is made with the baseball at position 1604c. Note that the set of data samples 1630 includes data samples 1630a and 1630b at relatively close proximity to each other in profile 1602. This indicates a deceleration (e.g., a slight, but detectable deceleration) in the bat when it hits the baseball. Thus, motion analyzer 1666 can analyze motion to determine motion-centric events, such as striking a baseball, striking a golf ball, or kicking a soccer ball. Data regarding the motion-centric events can be stored in database 1670 for additional analysis or archiving purposes, for example.

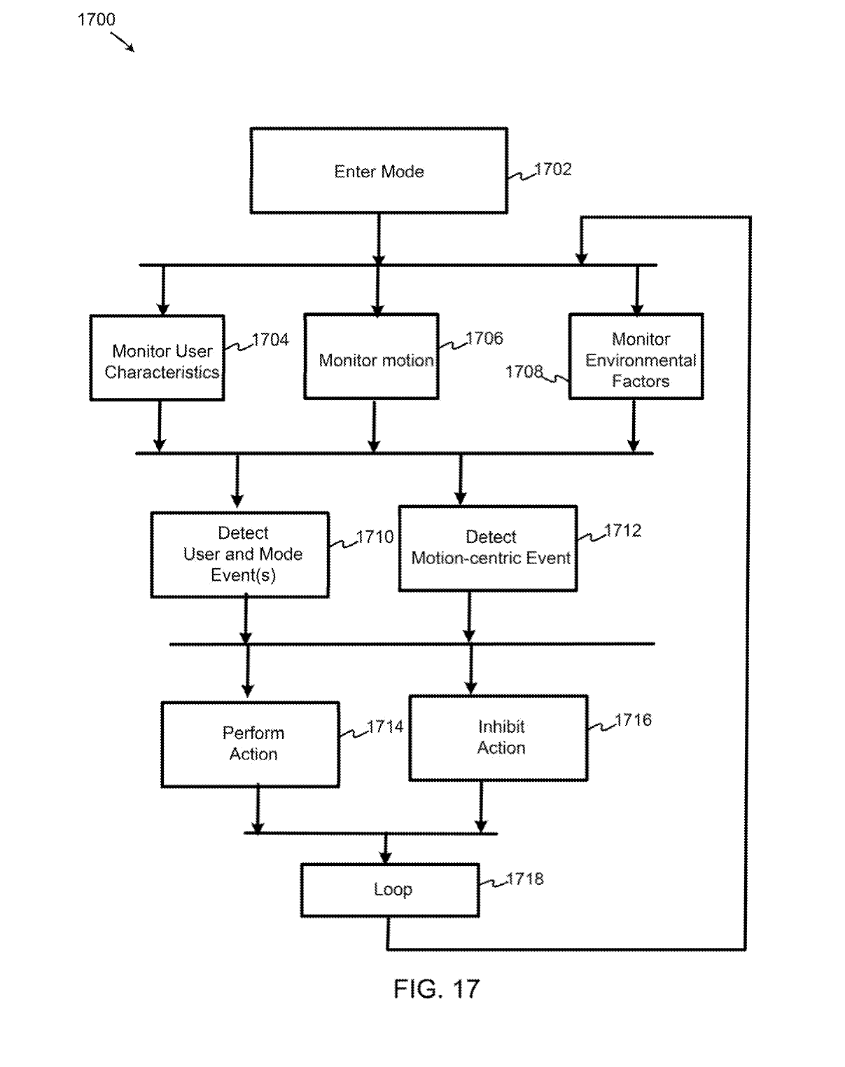

FIG. 17 illustrates action and event processing during a mode of operation in accordance with various embodiments. At 1702, the strapband enters a mode of operation. During a certain mode, a controller (e.g., a mode controller) can be configured to monitor user characteristics at 1704 relevant to the mode, as well as relevant motion at 1706 and environmental factors at 1708. The logic of the strapband can operate to detect user and mode-related events at 1710, as well as motion-centric events at 1712. Optionally, upon detection of an event, the logic of the strapband can perform an action at 1714 or inhibit an action at 1716, and continue to loop at 1718 during the activity or mode.

To illustrate action and event processing of a strapband, consider the following examples. First, consider a person is performing an activity of running or jogging, and enters an active mode at 1702. The logic of the strapband analyzes user characteristics at 1704, such as sleep patterns, and determines that the person has been getting less than a normal amount of sleep for the last few days, and that the person's heart rate indicates the user is undergoing strenuous exercise as confirmed by detected motion in 1706. Further, the logic determines a large number of wireless signals, indicating a populated area, such as along a busy street. Next, the logic detects an incoming call to the user's headset at 1710. Given the state of the user, the logic suppresses the call at 1716 to ensure that the user is not distracted and thus not endangered.

As a second example, consider a person is performing an activity of sleeping and has entered a sleep mode at 1702. The logic of the strapband analyzes user characteristics at 1704, such as heart rate, body temperature, and other user characteristics relevant to the determination whether the person is in REM sleep. Further, the person's motion has decreased sufficiently to match that typical of periods of deep or REM sleep as confirmed by detected motion (or lack thereof) at 1706. Environmental factors indicate a relatively dark room at 1708. Upon determination that the user is in REM sleep, as an event, at 1710, the logic of the strapband inhibits an alarm at 1716 set to wake the user until REM sleep is over. This process loops at 1718 until the user is out of REM sleep, when the alarm can be performed subsequently at 1714. In one example, the alarm is implemented as a vibration generated by the strapband. Note that the strapband can inhibit the alarm features of a mobile phone, as the strapband can communicate an alarm disable signal to the mobile phone.

In at least some examples, the structures and/or functions of any of the above-described features can be implemented in software, hardware, firmware, circuitry, or a combination thereof. Note that the structures and constituent elements above, as well as their functionality, may be aggregated with one or more other structures or elements. Alternatively, the elements and their functionality may be subdivided into constituent sub-elements, if any. As software, the above-described techniques may be implemented using various types of programming or formatting languages, frameworks, syntax, applications, protocols, objects, or techniques. As hardware and/or firmware, the above-described techniques may be implemented using various types of programming or integrated circuit design languages, including hardware description languages, such as any register transfer language (“RTL”) configured to design field-programmable gate arrays (“FPGAs”), application-specific integrated circuits (“ASICs”), or any other type of integrated circuit. These can be varied and are not limited to the examples or descriptions provided.

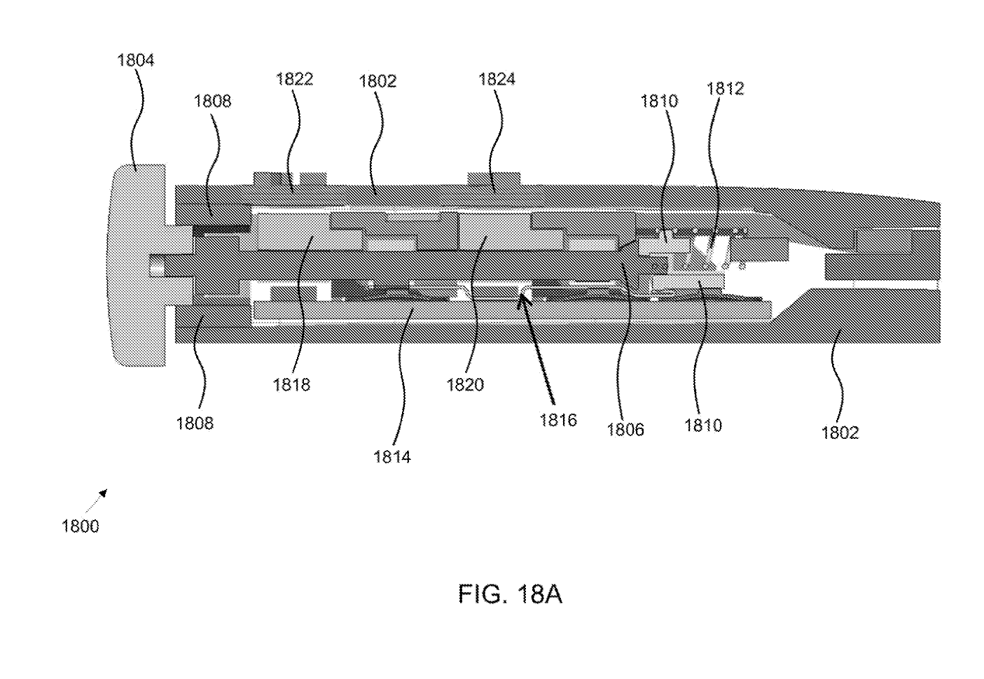

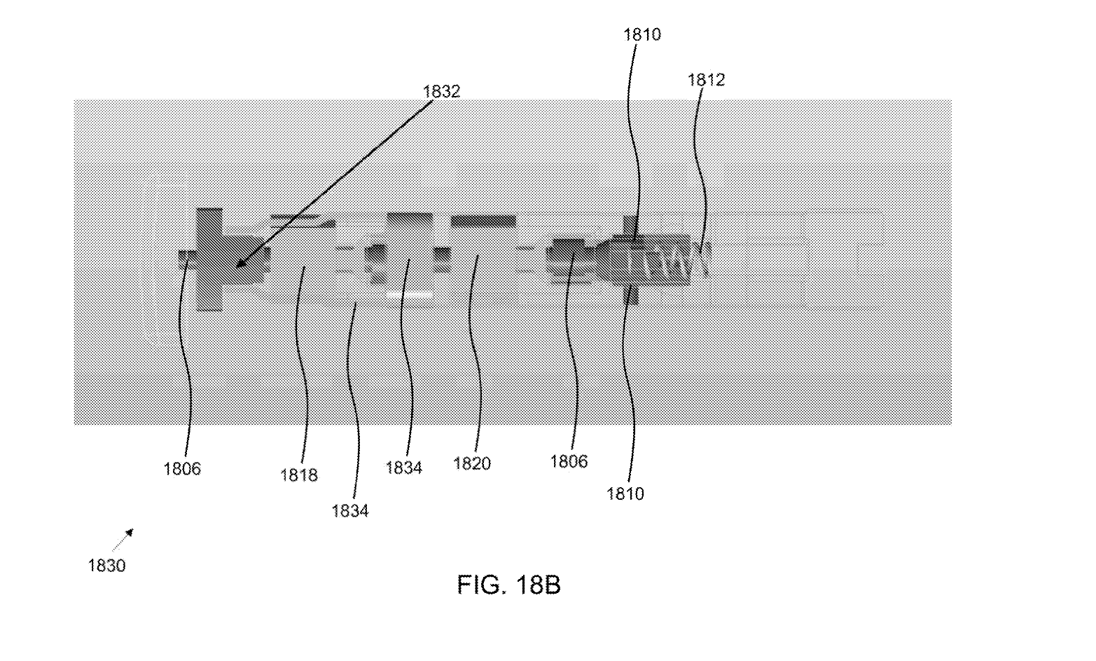

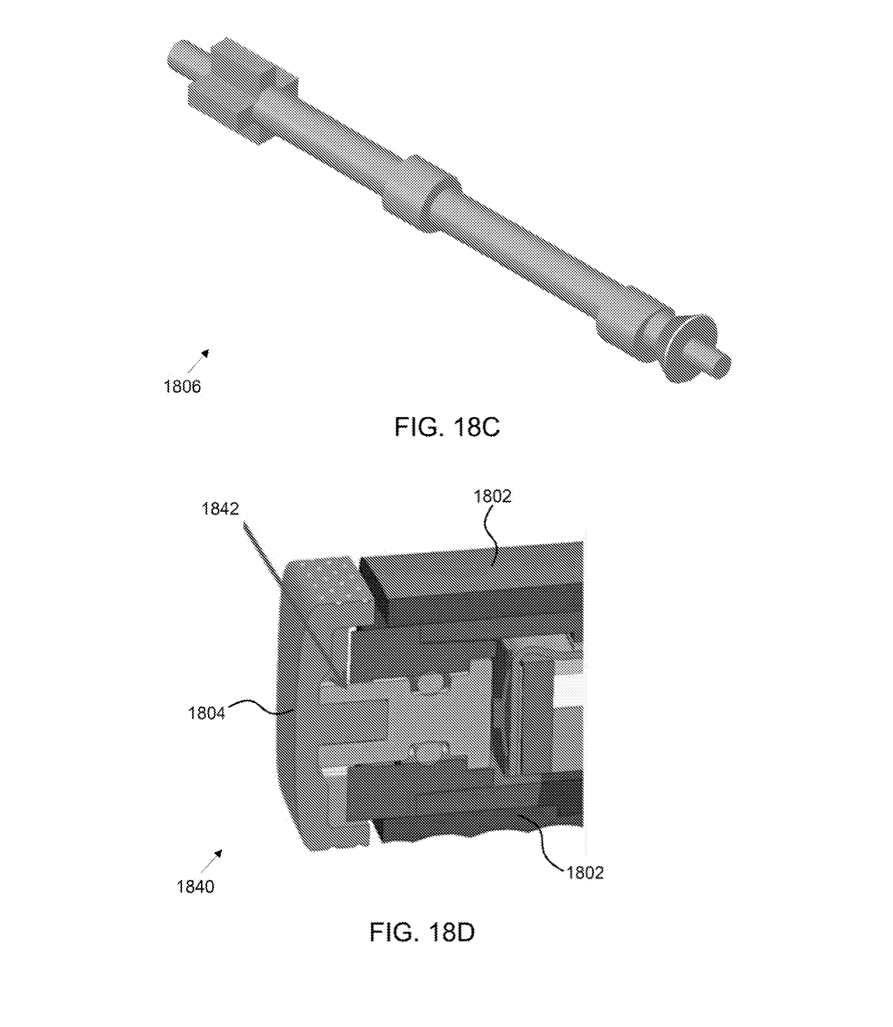

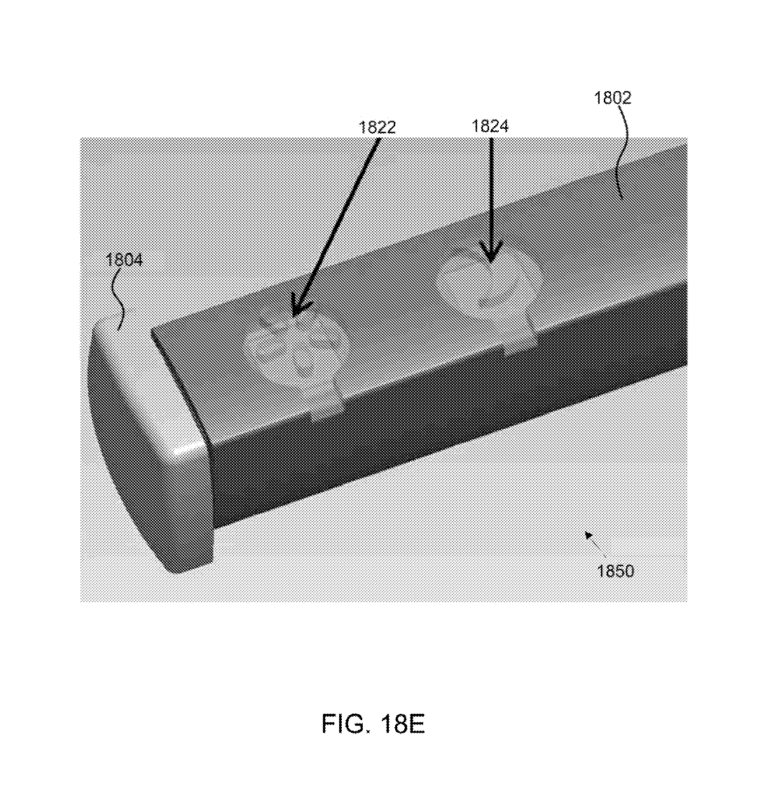

FIG. 18A illustrates an exemplary wearable device for sensory user interface. Here, a cross-sectional view of wearable device 1800 includes housing 1802, switch 1804, switch rod 1806, switch seal 1808, pivot arm 1810, spring 1812, printed circuit board (hereafter “PCB”) 1814, support 1816, light pipes 1818-1820, and light windows 1822-1824. In some examples, wearable device 1800 may be implemented as part of band 900 (FIG. 9A), providing a user interface for a user to interact, manage, or otherwise manipulate controls for a data-capable strapband. As shown, when switch 1804 is depressed and stopped by switch seal 1808, switch rod 1806 may be configured to mechanically engage pivot arm 1810 and cause electrical contact with one or more elements on PCB 1814. In an alternative example, pivot arm 1810 may cause light to be selectively reflected back, depending on the position of pivot arm 1810, to PCB 1814, which may comprise an optical transmitter/receiver to detect the reflection and to report back different rotational positions of pivot arm 1810. In another alternative example, pivot arm 1810 may comprise magnets, which may be brought into, and out of, proximity with one or more magnetic field sensor on PCB 1814 indicating different rotational positions of switch 1804. In other examples, switch 1804 may be configured to rotate and cause electrical contact with other elements on PCB 1814. Spring 1812 is configured to return switch rod 1806 and button 1804 to a recoiled position to await another user input (e.g., depression of switch 1804). In some examples, light sources (e.g., LED 224 (FIG. 2A)) may be mounted on PCB 1814 and, using light pipes 1818 and 1820 provide illuminated displays through light windows 1822 and 1824. Further, light windows 1822 and 1824 may be implemented as rotating switches that are translucent, transparent, or opaque and, when rotated, emit light from different features that visually indicate when a different function, mode, or operation is present. In other examples, wearable device 1800 may be implemented differently and is not limited to those provided.

FIG. 18B illustrates an alternative exemplary wearable device for sensory user interface. Here, a cross-sectional view of an alternative wearable device 1830 includes switch rod 1806, pivot arm 1810, spring 1812, light pipes 1818-1820, switch seal 1832, and detents 1834. In some examples, switch seal 1832 may be configured differently than as shown in FIG. 18A, providing a flush surface against which switch 1804 (FIG. 18A) may be depressed until stopped by detents 1834. Further, switch seal 1832 may be formed using material that is waterproof, water-resistant, or otherwise able to prevent the intrusion of undesired materials, chemicals, or liquids into the interior cavity of wearable device 1830. In other examples, wearable device 1830 may be configured, designed, formed, fabricated, function, or otherwise implemented differently and is not limited to the features, functions, and structures shown.

FIG. 18C illustrates an exemplary switch rod to be used with an exemplary wearable device. Here, a perspective view of switch rod 1806, which may be configured to act as a shaft or piston that, when depressed using switch 1804 (FIG. 18A), engages pivot arm 1810 (FIG. 18A) and moves into electrical contact one or more components on PCB 1814. Limits on the rotation or movement of switch rod 1806 may be provided by various types of mechanical structures and are not limited to any examples shown and described.

FIG. 18D illustrates an exemplary switch for use with an exemplary wearable device. Here, a distal end of wearable device 1840 is shown including housing 1802, switch 1804, and concentric seal 1842. As an alternative design, concentric seal 1842 may be implemented to provide greater connectivity between switch 1804 and detents 1834 (not shown; FIG. 18B). As shown, a concentric well in concentric seal 1842 may be configured to receive switch 1804 and, when depressed, engage switch rod 1806 (not shown; FIG. 18A). In other examples, wearable device 1840 and the above-described elements may be varied in function, structure, design, implementation, or other aspects and are not limited to those shown.

FIG. 18E illustrates an exemplary sensory user interface. Here, wearable device 1850 includes housing 1802, switch 1804, and light windows 1822-1824. In some examples, light windows 1822-1824 may be implemented using various designs, shapes, or features in order to permit light to emanate from, for example, LEDs mounted on PCB 1814. Further, light windows 1822-1824 may also be implemented as rotating switches that, when turned to a given orientation, provide a visual indication of a function, mode, activity, state, or operation being performed. In other examples, wearable device 1850 and the above-described elements may be implemented differently in design, function, or structure, and are not limited to those shown.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A wearable device, comprising:
a sensor coupled to the wearable device, the sensor being configured to sense at least one sensory input;
a processor configured to transform the at least one sensory input to data, the data being processed by an application configured to generate information associated with an activity during which the wearable device is worn; and
a communications facility coupled to the wearable device and configured to transfer the data between the wearable device and another device, the data being configured to be presented on a user interface during the activity.

2. The wearable device of claim 1, further comprising a motion matcher configured to capture other data representative of a motion.

3. The wearable device of claim 2, wherein the other data representative of the motion is used to identify another activity associated with the motion.

4. The wearable device of claim 1, wherein the wearable device is configured to transition from a first mode of operation to a second mode of operation as a function of the activity.

5. The wearable device of claim 1, wherein the sensor is an accelerometer configured to detect a motion, the motion being converted to other data by the accelerometer.

6. The wearable device of claim 1, wherein the sensor is configured to measure a temperature.

7. The wearable device of claim 1, wherein the sensor is configured to measure a first temperature and a second temperature.

8. The wearable device of claim 1, wherein a temperature differential between the first temperature and the second temperature is determined by the processor.

9. The wearable device of claim 1, wherein the sensor is configured to measure galvanic skin response.

10. The wearable device of claim 1, further comprising a housing that is flexible and configured to adapt to an anatomical body around which the wearable device is worn.

11. The wearable device of claim 1, wherein the data is used by the processor to determine a caloric burn rate.

12. The wearable device of claim 1, wherein the application is hosted remotely from the wearable device, the data being transferred between the wearable device and the application using a data communication link.

13. The wearable device of claim 1, wherein the data communication link is based on a Bluetooth protocol.

14. The wearable device of claim 1, wherein the data communication link is wireless.

15. The wearable device of claim 1, wherein the data is transferred between the wearable device and the application using a wired data communication.

16. A wearable device, comprising:
a sensor coupled to a framework having a housing comprised of one or more moldings, the sensor being configured to sense at least one sensory input;
a processor configured to transform the at least one sensory input to data during an activity in which the wearable device is worn; and
a communications facility coupled to the wearable device and configured to transfer the data between the wearable device and another device during the activity, the data being configured to be presented on a user interface.

17. The wearable device of claim 16, wherein the processor is configured to generate a control signal to a feedback mechanism configured to generate a sensory output.

18. The wearable device of claim 16, further comprising a visual indicator.

19. The wearable device of claim 18, wherein the visual indicator is configured to provide an indication of health.

20. The wearable device of claim 18, wherein the visual indicator comprises one or more buttons.

21. The wearable device of claim 18, wherein the visual indicator has one or more positions.

22. The wearable device of claim 18, wherein the visual indicator indicates state activity.

23. The wearable device of claim 18, wherein the visual indicator is a rotating structure.

24. The wearable device of claim 18, wherein the another device is a mobile communications device.

25. The wearable device of claim 16, wherein an alert configured to simulate behavior associated with the wearable device is generated by the wearable device during an activity state.

26. The wearable device of claim 16, wherein the data is used to monitor a mood.

27. The wearable device of claim 16, wherein the sensory input is a GPS signal.

28. The wearable device of claim 16, wherein the user interface is further configured to present other data associated with another wearable device, the other data indicating another activity in which the another wearable device is worn.

29. The wearable device of claim 16, wherein the user interface is further configured to present other data associated with another wearable device, the other data indicating an activity state of the another wearable device.

30. The wearable device of claim 16, wherein the data is evaluated to generate a representation of health, the representation being configured to be displayed on the user interface.

31. The wearable device of claim 16, wherein the data is evaluated to generate a representation of wellness, the representation being configured to be displayed on the user interface.

32. A platform, comprising:
a wearable device having one or more sensors configured to detect one or more sensory inputs, the one or more sensory inputs being transformed into data associated with a state; and
an application configured to evaluate the data from the one or more sensory inputs, to perform a comparison of the data to other data associated with the wearable device and stored in a database, and to generate a representation of the state based on a comparison of the data to the other data.

33. The platform of claim 32, wherein the wearable device is further configured to generate an alert based on one or more parameters stored in a memory associated with the wearable device.

34. The platform of claim 32, wherein the wearable device is further configured to generate an alert based on one or more parameters stored in a memory associated with the wearable device when the representation exceeds the one or more parameters.

35. A platform, comprising:
a wearable device configured to receive a sensory input, the sensory input being transformed into data by a sensor in data communication with the wearable device; and
an application configured to evaluate the data to generate a representation of a state, the representation being used to determine one or more recommendations associated with the state.

36. The platform of claim 35, wherein the application is installed on the wearable device.

37. The platform of claim 35, wherein the application is installed at a location remote from the wearable device.

38. The platform of claim 35, wherein the application further comprises a user interface configured to receive another input associated with the representation.
